SEQUENCE LISTING (27 sequences)

SEQ ID NO: 1 - DNA, 2271 bp, Homo sapiens
ctggctggtaccatgcagctcccactggccctgtgtctcgtctgcctgctggtacacacagccttccgtgtagtggagggccaggggtggcaggcgttcaagaatgatgccacggaaatcatccccgagctcggagagtaccccgagcctccaccggagctggagaacaacaagaccatgaaccgggcggagaacggagggcggcctccccaccacccctttgagaccaaagacgtgtccgagtacagctgccgcgagctgcacttcacccgctacgtgaccgatgggccgtgccgcagcgccaagccggtcaccgagctggtgtgctccggccagtgcggcccggcgcgcctgctgcccaacgccatcggccgcggcaagtggtggcgacctagtgggcccgacttccgctgcatccccgaccgctaccgcgcgcagcgcgtgcagctgctgtgtcccggtggtgaggcgccgcgcgcgcgcaaggtgcgcctggtggcctcgtgcaagtgcaagcgcctcacccgcttccacaaccagtcggagctcaaggacttcgggaccgaggccgctcggccgcagaagggccggaagccgcggccccgcgcccggagcgccaaagccaaccaggccgagctggagaacgcctactagagcccgcccgcgcccctccccaccggcgggcgccccggccctgaacccgcgccccacatttctgtcctctgcgcgtggtttgattgtttatatttcattgtaaatgcctgcaacccagggcagggggctgagaccttccaggccctgaggaatcccgggcgccggcaaggcccccctcagcccgccagctgaggggtcccacggggcaggggagggaattgagagtcacagacactgagccacgcagccccgcctctggggccgcctacctttgctggtcccacttcagaggaggcagaaatggaagcattttcaccgccctggggttttaagggagcggtgtgggagtgggaaagtccagggactggttaagaaagttggataagattcccccttgcacctcgctgcccatcagaaagcctgaggcgtgcccagagcacaagactgggggcaactgtagatgtggtttctagtcctggctctgccactaacttgctgtgtaaccttgaactacacaattctccttcgggacctcaatttccactttgtaaaatgagggtggaggtgggaataggatctcgaggagactattggcatatgattccaaggactccagtgccttttgaatgggcagaggtgagagagagagagagaaagagagagaatgaatgcagttgcattgattcagtgccaaggtcacttccagaattcagagttgtgatgctctcttctgacagccaaagatgaaaaacaaacagaaaaaaaaaagtaaagagtctatttatggctgacatatttacggctgacaaactcctggaagaagctatgctgcttcccagcctggcttccccggatgtttggctacctccacccctccatctcaaagaaataacatcatccattggggtagaaaaggagagggtccgagggtggtgggagggatagaaatcacatccgccccaacttcccaaagagcagcatccctcccccgacccatagccatgttttaaagtcaccttccgaagagaagtgaaaggttcaaggacactggccttgcaggcccgagggagcagccatcacaaactcacagaccagcacatcccttttgagacaccgccttctgcccaccactcacggacacatttctgcctagaaaacagcttcttactgctcttacatgtgatggcatatcttacactaaaagaatattattgggggaaaaactacaagtgctgtacatatgctgagaaactgcagagcataatactgccacccaaaaatctttttgaaaatcatttccagacaacctcttactttctgtgtagtttttaattgttaaaaaaaaaaagttttaaacagaagcacatgacatatgaaagcctgcaggactggtcgtttttttggcaattcttccacgtgggacttgtccacaagaatgaaagtagtggtttttaaagagttaagttacatatttattttctcacttaagttatttatgcaaaagtttttcttgtagagaatgacaatgttaatattgctttatgaattaacagtctgttcttccagagtccagagacattgttaataaagacaatgaatcatgaaaaaaaaaaaaaa

SEQ ID NO: 2 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 3 - DNA, 7099 bp, Homo sapiens
ggagctatggaaggcttttgagcaggggagtgatacaatgagaacagcattttggaagccagcatggaggggaggctgctaaaaggatacaaggctgaggttattaccatgccctgggtgaagaaatgcggtctgggtttggaaaggaagggataaacatgacttttcaaaggaagacataattgccacaagggccaccagagagcagtcaaaggtgtgtccaaggttggagctctagtgaccaagagaagggtgtcaactttgacagaaatgacgtcatgtgtggttgacaaaatgagaccacatcctttcggtcatgattcattgtctttattaacaatgtctctggactctggaagaacagactgttaattcataaagcaatattaacattgtcattctctacaagaaaaacttttgcataaataacttaagtgagaaaataaatatgtaacttaactctttaaaaaccactactttcattcttgtggacaagtcccacgtggaagaattgccaaaaaaacgaccagtcctgcaggctttcatatgtcatgtgcttctgtttaaaactttttttttttaacaattaaaaactacacagaaagtaagaggttgtctggaaatgattttcaaaaagatttttgggtggcagctattatgctctgcagtttctcagcatatgtacagcacttgtagtttttcccccaataatattcttttagtgtaagatatgccatcacatgtaagagcagtaagaagctgttttctaggcagaaatgtgtccgtgagtggtgggcagaaggcggtgtctcaaaagggatgtgctggtctgtgagtttgtgatggctgctccctcgggcctgcaaggccagtgtccttgaacctttcacttctcttcggaaggtgactttaaaacatggctatgggtcgggggagggatgctgctctttgggaagttggggcggatgtgatttctatccctcccaccaccctcggaccctctccttttctaccccaatggatgatgttatttctttgagatggaggggtggaggtagccaaacatccggggaagccaggctgggaagcagcatagcttcttccaggagtttgtcagccgtaaatatgtcagccataaatagactctttacttttttttttctgtttgtttttcatctttggctgtcagaagagagcatcacaactctgaattctggaagtgaccttggcactgaatcaatgcaactgcattcattctctctctttctctctctctctctcacctctgcccattcaaaaggcactggagtccttggaatcatatgccaatagtctcctcgagatcctattcccacctccaccctcattttacaaagtggaaattgaggtcccgaaggagaattgtgtagttcaaggttacacagcaagttagtggcagagccaggactagaaaccacatctacagttgcccccagtcttgtgctctgggcacgcctcaggctttctgatgggcagcgaggtgcaagggggaatcttatccaactttcttaaccagtccctggactttcccactcccacaccgctcccttaaaaccccagggcggtgaaaatgcttccatttctgcctcctctgaagtgggaccagcaaaggtaggcggccccagaggcggggctgcgtggctcagtgtctgtgactctcaattccctcccctgccccgtgggacccctcagctggcgggctgaggggggccttgccggcgcccgggattcctcagggcctggaaggtctcagccccctgccctgggttgcaggcatttacaatgaaatataaacaatcaaaccacgcgcagaggacagaaatgtggggcgcgggttcagggccggggcgcccgccggtggggaggggcgcgggcgggctctagtaggcgttctccagctcggcctggttggctttggcgctccgggcgcggggccgcggcttccggcccttctgcggccgagcggcctcggtcccgaagtccttgagctccgactggttgtggaagcgggtgaggcgcttgcacttgcacgaggccaccaggcgcaccttgcgcgcgcgcggcgcctcaccaccgggacacagcagctgcacgcgctgcgcgcggtagcggtcggggatgcagcggaagtcgggcccactaggtcgccaccacttgccgcggccgatggcgttgggcagcaggcgcgccgggccgcactggccggagcacaccagctcggtgaccggcttggcgctgcggcacggcccatcggtcacgtagcgggtgaagtgcagctcgcggcagctgtactcggacacgtctgtggagagaggcgcgcgtgagggtggccgcccgcctggccacccctgccctggaccggcccgtctctctccaccccagccctgcttttgccaagcctgtctccagaggcttttgcccctgaacatctattgcaggaaggtcccagatgctctagagctggtggaaagctctcctgcgcaccctgtcccctcggagccaaatgactgctggggttcaaacaccagggctccatcgctcactgcaggtgtcatcttgggcaagttccttaacctctctcttcttcctgtctgcaaaatgggccgccccaagcattgcgagttaatccattacacacgcaatgcagaaaataccatttggcacatatagctgaaagcgtcggctgttatgtattattattatcatttctcttctggggtcttcttatgatttaagccacacttgtgccgcagggtccctggcctgggcagggtaagtactcagcacaccttctgcctggctctggcttctcccaaattccttttccagccacttgggaggggtggacaggggatggagaaaggagtagaggttaattgaaggtaataaatcagggcagctgctgtggaggcgagggactgaaggggaggtgatcaggacaggaattcattagacttgaaaggaatcaaaggcagtgtgtccaaccccctcattatcaatgagaatactgaggcccagccagagaaaagaacttgcccaaaaggctgggctgcatctccggactccacttccctggggtcccagcaccttttggctccggcaagagtggaggctctgcgttcgagcatccatgggaggctgggctgggggatggagctgagggctgctttttactgtcgttactttcctaccataggggccctgagctctgacccaaaacctacaggttcagctgccacaaacattgacctggctctctgctgggcacatttcttgttctgtgttatctgaatgggcgcctcatggtgatactccctgggcaggcagagattgggctccgaggcctggcctgatcccctgcacacaccttgaccatgtctcctatggtggacttagatgggtgggagctttttaaatagctggtcgtcgtgtgagtaccagtccacccccaaaggtttcatggggatagttccaatgtgacaagaaagaatgaaaaatagtcttaagaccaaaccagagaggggacagagaaggccaccccccacccccactctgaagagcaagcacagcaggggagatgtggccactgccatcgtctggggctgctttctggtctgtgggctggtttgcatcctcacctctgcagggctggaatgtgtgtgtggtcaggggggcatttgcccagggaagggggcattttgcggcttttcgagaatagcgcccctaggccaggcacttcctctgcagatcatgactttgttttgagcatgtggcttcctcaggggctgtggcctctcgtgggagtccagcaaggtggacattttgtgtttgggttccttacagcccttcaggcagtcgctggagcccagctgaccttgcctagaaggggagctgtggggggaggcgcccttgtcctccattttgaagacggagggtcctggctggcctcccatgcagttgccctttttagcaccccatagtcccaacaggttgtgaggcggggagggctggggtccaggagcctgggcagtggacttctctgccacctggataatggccagaggacagggaagctcttagtctgaagtgtcatttcagacgcccccatggcctcaggattcctgccaattatagagccagatgcacactgtcacctgggcttctctgtgcaggcctgaccaggaacccagggttcctcttccttctccctcccctggaatgaggcaaggttgggactggggtggctgctgaggacagatgtccctactcctgcacagaggatgctgctctcccctccagggcctggactcagctgcacccatgtcaggggctaaaactgtgccgaaaactcccattccttcaagtgccagctccctgggcaggccatgggcaatgcacactgcaggggtgcctctctggccttcctccccactgctttgtggagatctgtcccccacacccccccacacaccagggctatcttgcagttggatcaaaccaggttcacacctcagctcttctcactaccaagttactgagtgcctggaacctgtttcttccccttcgagctgggggttcacctcctggggattccatgatgtgggtctgtgtggtggggacagccaccaccagcatagagtgtccggtgccccacaggtgctgttctgtggatgggaggctcccttaccttgttcccttcccagtggtaccagcagctggacgtggctcctatattcccaggaggagattctttgaaaccttttcttttctgagcccctgagcctgttctgatgccctgatgccataccctcaccaccccagcgtcctccctgccctgggctctcaccagcgtctgttgcctcatgcctctgcacctgaatttgaaggctgcaccagcgagccattcttcccccacctccaggcaagactgttcctcgaccagtgctggcagaggccagcaatcttcactgggtctaccccagtcttccaagcctcccaaagagaagtttctaaaacctccccaggatcttttactaagaattctctcctccaccccatacctttggtctcaaaggggtggtggggaggccgccctccgttctccgcccggttcatggtcttgttgttctccagctccggtggaggctcggggtactctccgagctcggggatgatttccgtggcatcattcttgaacgcctgccacccctggccctccactacacggaaggctgtgtgtaccagcaggcagacgagacacagggccagtgggagctgcatggtaccagccagaggagggcacgccaccttccagtagcacaggctctggtctccagccgagacacggtcgcctttttaaagcccctctctgcttcatgccagccaatgaggacaggctggggcagggctggtcccatgtttccctcagccccggagggagggaaaggggtgtgctcagagcaaaccctccccaaagacttctcctctagctcagcaaacttccaaattgctgctggcactcccaggtgacccagagagagggggcgtgtgaggcaaggcccaagcctgctctccggcacctcccacgtgctggcggctgtggttttcagatatcaaaatgagctccggcttttaattgtctgtctccctgggccctcgggcattctcaaaacaaactgtggacccgctcaacaaagaaaatgtgcagttctgagtgcttcgcggggctgagcccggcctattccatttctcaggccaagttcatttgtcaccccaccaacaaatggcccaaggtgtttctgtttgacccccatagacccagccagaggcaaaaagatgcttttaaagaaatcccattggtcccaactccacaagtctcagcattcattgtcctaaccaagaaagcctctcttggtattctctggaaggcaagagagagaactagagggtcagagcaagggtgcccgcttgggagagagggcaggattctggttacaaatcgaggatgcgctggtcactcaggctcctgtgatggacagcacagaggaaatcccagctcatgctcctcagggatcctgtgcactgtgacaggcacttctgagccagggcactcactgtggctcgcagggtgatgtgctgaagacagtcatttccccaaccagtctgtgtgtgtgagaagacaacaccaaattcctttccttagcaaaagccacagccagcgtttttagtgctgacattttattttcttgtcttccagctggttacatttttcaaaaacagttttgagtcctgtatggaaagcttcattggtgcacaccacaggatgtgttccatgaagccatcccaggaccaactggtcacaaagaagctaagagaaaatgctgccttggccctgcatataatgagccacacagggccggggcggggggcgggggcgcaggtagaggcgtgggggagaaggattcacacctgaggtgcaagaagagaaaggaaggccagaggaatctttcattgaagacagattgtttgaggacaagcagctcctgtgaagaggctgcccagatcctctggtattttaactgggcaatgaatcagcatgccccacctgcagagcctctgcaagtcctttgccacctgcctcaacttgcaagtcgctgtgtccatctgcagccgactgtcctttccatcatccatcatccgacacatgaatgcagctgctctgacagttttacaagccagacagaagccccctcaggaagggccctctctgatacctgcttcctaagtcagtttccttcagggcagatgaaaacagctgtggccattgttgggggtgatctatctctcccagcagagccggtagtgttgtgagaagctggccctccgcaccacctccccttgtctatgaccctccagcctccttttcccttgctctggaatgtggactgttaaccctctcatgaccagacgactgccttcttgcaatggacttgggagagagcagttctgatgcctgggttccttgagcaggtaggatggatgcttatagccccacctgctg

SEQ ID NO: 4 - DNA, 27 bp, Artificial Sequence
  Description of Artificial Sequence Primer
tctgtttgtttttcatctttggctgtc

SEQ ID NO: 5 - DNA, 23 bp, Artificial Sequence
  Description of Artificial Sequence Primer
accttccaggccctgaggaatcc

SEQ ID NO: 6 - DNA, 23 bp, Artificial Sequence
  Description of Artificial Sequence Primer
cactggccggagcacaccagctc

SEQ ID NO: 7 - DNA, 21 bp, Artificial Sequence
  Description of Artificial Sequence Primer
accctgcggcacaagtgtggc

SEQ ID NO: 8 - DNA, 20 bp, Artificial Sequence
  Description of Artificial Sequence Primer
ctgagctctgacccaaaacc

SEQ ID NO: 9 - DNA, 19 bp, Artificial Sequence
  Description of Artificial Sequence Primer
caccttgctggactcccac

SEQ ID NO: 10 - DNA, 22 bp, Artificial Sequence
  Description of Artificial Sequence Primer
ggcacttcctctgcagatcatg

SEQ ID NO: 11 - DNA, 21 bp, Artificial Sequence
  Description of Artificial Sequence Primer
cagggagctggcacttgaagg

SEQ ID NO: 12 - DNA, 21 bp, Artificial Sequence
  Description of Artificial Sequence Primer
cctccagggcctggactcagc

SEQ ID NO: 13 - DNA, 23 bp, Artificial Sequence
  Description of Artificial Sequence Primer
gaagaatggctcgctggtcgagc

SEQ ID NO: 14 - DNA, 21 bp, Artificial Sequence
  Description of Artificial Sequence Primer
gctctcaccagcgtctgttgc

SEQ ID NO: 15 - DNA, 21 bp, Artificial Sequence
  Description of Artificial Sequence Primer
gggagggtttgctctgagcac

SEQ ID NO: 16 - DNA, 20 bp, Artificial Sequence
  Description of Artificial Sequence Primer
ggacaggctggggcagggct

SEQ ID NO: 17 - DNA, 23 bp, Artificial Sequence
  Description of Artificial Sequence Primer
gaatcctgccctctctcccaagc

SEQ ID NO: 18 - DNA, 21 bp, Artificial Sequence
  Description of Artificial Sequence Primer
aagagtctatttatggctgac

SEQ ID NO: 19 - DNA, 20 bp, Artificial Sequence
  Description of Artificial Sequence Primer
tcctgcaggctttcatatgt

SEQ ID NO: 20 - DNA, 22 bp, Artificial Sequence
  Description of Artificial Sequence Primer
gttttaaacagaagcacatgac

SEQ ID NO: 21 - DNA, 21 bp, Artificial Sequence
  Description of Artificial Sequence Primer
gagtgatacaatgagaacagc

SEQ ID NO: 22 - DNA, 20 bp, Artificial Sequence
  Description of Artificial Sequence Primer
ggacaggctggggcagggct

SEQ ID NO: 23 - DNA, 23 bp, Artificial Sequence
  Description of Artificial Sequence Primer
gaatcctgccctctctcccaagc

SEQ ID NO: 24 - DNA, 21 bp, Artificial Sequence
  Description of Artificial Sequence Primer
ctcttggtattctctggaagg

SEQ ID NO: 25 - DNA, 19 bp, Artificial Sequence
  Description of Artificial Sequence Primer
gcctcttcacaggagctgc

SEQ ID NO: 26 - DNA, 20 bp, Artificial Sequence
  Description of Artificial Sequence Primer
aggattcacacctgaggtgc

SEQ ID NO: 27 - DNA, 21 bp, Artificial Sequence
  Description of Artificial Sequence Primer
ataagcatccatcctacctgc

(19) United States
(12) Patent Application Publication
(10) Pub. No.: US 2004/0132021 A1
(43) Pub. Date: Jul. 8, 2004

(54) Osteolevin gene polymorphisms

(76) Inventors: Wendy Balemans, Antwerpen(Wilrijk) (BE); Martin Ebeling, Grenzach-wyhlen (DE); Dorothee Foernzler, Lenzburg (CH); Neela Patel, Belmont, CA (US); Wim Van Hul, Hever (BE)

Correspondence Address: George W Johnston, Hoffmann La Roche Inc, 340 Kingsland Street, Nutley, NJ, 07110, US

(21) Appl. No.: 10/311,490

(22) PCT Filed: Jun. 15, 2001
(86) PCT No.: PCT/EP01/06795

(30) Foreign Application Priority Data
Jun. 19, 2000 (EP) 00112867.7

(51) Int. Cl. C12Q 1/68

(52) U.S. Cl. 435/6

(57) ABSTRACT

The present inventon relates generally to genetic polymorphisms in the Van Buchem-scierosteosis disease region. In particular, the present invention relates to genetic polymorphisms in the Van Buchem-sclerosteosis disease region that are associated with disorders resulting in either net excess bone formation or insufficient bone formation in humans. Furthermore, isolated nucleic acid molecules encoding human osteolevin are provided. Osteolevin polypeptides are also provided as are vectors, host cells and recombinant methods for producing the same.

The present invention relates generally to genetic polymorphisms in the Van Buchem-sclerosteosis disease region. In particular, the present invention relates to genetic polymorphisms in the Van Buchem-sclerosteosis disease region that are associated with disorders resulting in either net excess bone formation or insufficient bone formation in humans.

Over 2000 human pathological syndromes are known to result from DNA polymorphisms including insertions, deletions, duplications and nucleotide substitutions. Finding genetic polymorphisms in individuals and following these variations in families provides a means to confirm clinical diagnoses and to diagnose both predispositions and disease states in carriers, as well as preclinical and subdinical affected individuals. Counselling based upon accurate diagnoses allows patients to make informed decisions about potential parenting, ongoing pregnancy, and early intervention in affected individuals.

Polymorphisms associated with pathological syndromes are highly variable and, consequently, can be difficult to identify. Because multiple alleles within genes are common, one must distinguish disease-related alleles from neutral (non-disease-related) polymorphisms. Most alleles are neutral polymorphisms that produce indistinguishable, normally active gene products or express normally variable characteristics like eye color. In contrast, some polymorphic alleles are associated with clinical diseases such as sickle cell anemia. Moreover, the structure of disease-related polymorphisms are highly variable and may result from a single point mutation such as occurs in sickle cell anemia, or from the expansion of nucleotide repeats as occurs in fragile X syndrome and Huntington's chorea. Additionally, some polymorphic alleles may be associated with a phenotype which is manifested as a particular response to treatment with drug(s).

Once a polymorphism or region of interest is identified, a wide variety of technologies exist which may be employed to diagnose a disease or pre-disposition to the disease. Traditionally, the diagnosis of such syndromes relied upon enzyme activity testing, statistical analysis, or invasive diagnostic procedures. More recently, advances in DNA and related technologies including restriction fragment length polymorphism (RFLP) analysis, the polymerase chain reaction (PCR), and monoclonal or polyclonal antibody based assays provide rapid and highly accurate methods to screen for the presence of polymorphisms associated with heritable pathologies.

Genetic variants may be involved in a number of genetic disorders including pathologies associated with bone formation disorders such as Van Buchem's disease. Van Buchem disease (Hyperostosis Corticalis Generalisata; OMIM 239100) and sderosteosis (OMIM 269500) are two autosomal recessive conditions belonging to the group of endosteal hyperostoses and are both characterized by a generalized hyperostosis most pronounced in the skull (both the calvarium and the skull base) and the mandible. In addition the ribs, clavicle and the diaphyses of the long bones are sclerotic. Clinical complications include facial nerve palsy, sensorineural hearing loss and sometimes visual problems. Differential diagnosis between Van Buchem disease and sclerosteosis is mainly based on the presence of syndactyly and gigantism in sclerosteosis. Sclerosteosis also has in most cases a more severe phenotype. Genetic localization of the Van Buchem's disease gene in a Dutch family and the sclerosteosis disease gene in a Brazilian and an American family led to the assignment of both disease genes to the same region on chromosome 17q12-q21. The co-localization of both conditions to the same linkage interval supports the hypothesis that Van Buchem disease and sclerosteosis are caused by mutations in the same gene.

The identification and characterization of specific polymorphisms associated with heritable disorders resulting in either net excess bone formation or insufficient bone formation, such as osteoporosis and/or sclerosing diseases, is necessary for the design of informative diagnostic assays. By identifying specific regions in the human genome which contain disease related polymorphisms, statistical analysis of the prevalence and penetrance of the syndrome is possible. Further, as different statistical formulas are utilized for the assessment of autosomal recessive, autosomal dominant, and X-linked genetic diseases, the identification of the chromosomal location of the polymorphism is a crucial factor in the assessment of pedigree-related risk analysis. Such information allows accurate risk assessments to take into account 1) the number of different alleles at each gene locus, 2) the relative frequency of each allele in the Population (the most informative have more than one common allele), and 3) whether alleles are distributed randomly throughout the Population. As technologies for assessing the presence or absence of a specific polymorphism or polymorphic region are well developed, the primary limitation for diagnostic assays is the lack of information regarding polymorphisms associated with different pathologies.

What is needed in the art is the identification of regions in the human genome containing disease-associated polymorphisms. The identification of such regions allows for the design of informative assays and diagnostic tests for susceptibility factors associated with the occurrence of such syndromes. Informative assays which can detect the disease associated polymorphism will allow the accurate diagnosis of affected individuals and provide these individuals and health care professionals with the knowledge necessary to make informed decisions.

The present invention is directed to the identification of polymorphisms in the osteolevin gene (also referred to as SOST gene) which is located in the Van Buchem-sclerosteosis disease region of human chromosome 17, a region that is associated with abnormal bone formation. Polymorphisms in this region were identified by sequencing a large number of samples of DNAs from a diverse population of different ethnic origin. The present invention also relates to the use of the polymorphic regions disclosed herein for the diagnosis and assessment of osteoporosis or sclerosis. Since such pathologies can now be detected earlier (i.e., before overt symptoms appear) and more definitively, better treatment options will be available for those individuals identified as having disease-associated polymorphisms.

In one embodiment, the invention provides isolated nucleic acid molecules that encode osteolevin region polymorphisms. Isolated nucleic acid can include osteolevin region polymorphisms having the sequences identified above or having sequences that are complementary to these nucleic acid sequences, preferentially hybridize to them and remain stably bound to them under at least moderate, and optionally, under high stringency conditions.

In another embodiment, the invention provides a construct where the polymorphic osteolevin region sequences have been inserted in a vector to make a recombinant plasmid. A recombinant cell comprising such a plasmid inserted into a host cell is also provided.

In another embodiment, the present invention provides a recombinant osteolevin polypeptide and its variants due to the osteolevin region polymorphisms as well as isolated nudeic acid molecules encoding the osteolevin polypeptide and its variants.

In yet another embodiment, the invention provides antibodies capable of specifically binding a polymorphic epitope on a polypeptide encoded by the osteolevin gene and the region polymorphisms. Optionally, the antibody is a monoclonal antibody. In yet another embodiment, the invention provides animals having osteolevin region transgenes.

In other embodiments, the invention provides methods for screening for osteolevin region polymorphisms. In one embodiment, the invention provides a method for screening for a polymorphism associated with abnormal bone formation in a subject. The subject's DNA is analyzed for the presence or absence of the osteolevin polymorphism. The polymorphism which is an insertion, deletion, duplication, or base substitution is associated with abnormal bone formation. In a specific embodiment of this method the polymorphism may be one or several of the osteolevin polymorphisms disclosed herein. In a more specific embodiment of the invention, the presence of a polymorphism in the osteolevin nucleic acid sequence is determined by a differential nucleic acid analysis technique such as restriction fragment length polymorphism analysis, direct mass-analysis of PCR products using mass spectrometry, direct analysis of invasive cleavage products, extension-based techniques such as ARMS™ (amplification refractory mutation system), ALEX™ (amplification refractory mutation system linear extension) and COPS (competitive oligonudeotide priming system), OLA (oligonucleotide ligation assay), Invader assay, direct sequence analysis or polymerase chain reaction analysis.

In another embodiment, the invention provides a method for identifying a patient's susceptibility to pathologies associated with abnormal bone formation by determining the patient's osteolevin polymorphism pattern, comparing it to the wild type osteolevin pattern and then looking for differences indicative of a susceptibility to pathologies associated with abnormal bone formation. In a related embodiment, the invention provides a method of identifying a polymorphism associated with abnormal bone formation by comparing a osteolevin gene sequence isolated from a subject with abnormal bone formation to a known wild type osteolevin gene sequence and identifying recurrent polymorphisms. In specific embodiments of these methods, the presence of a polymorphism in osteolevin nudeic acid sequences is determined by a differential nucleic acid analysis technique such as restriction fragment length polymorphism analysis, direct mass-analysis of PCR products using mass spectrometry, direct analysis of invasive cleavage products, direct sequence analysis, extension based techniques such as ARMS™ (amplification refractory mutation system), ALEX™ (amplification refractory mutation system linear extension) and COPS (competitive oligonudeotide priming system), OLA (oligonucleotide ligation assay), Invader Assay, DNA chip analysis or polymerase chain reaction analysis.

Other embodiments of the invention include kits and articles of manufacture for use in the methods disclosed herein as well as assays for assessing the effects of candidate agents on the activity of genes from the Van Buchem-sclerosteosis disease region.

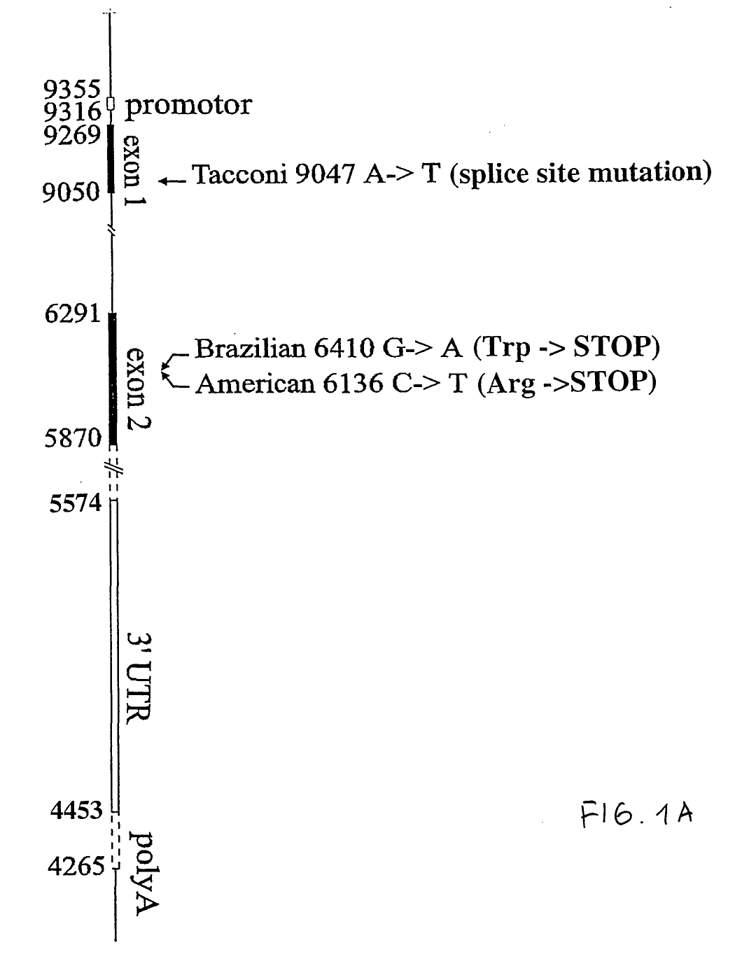

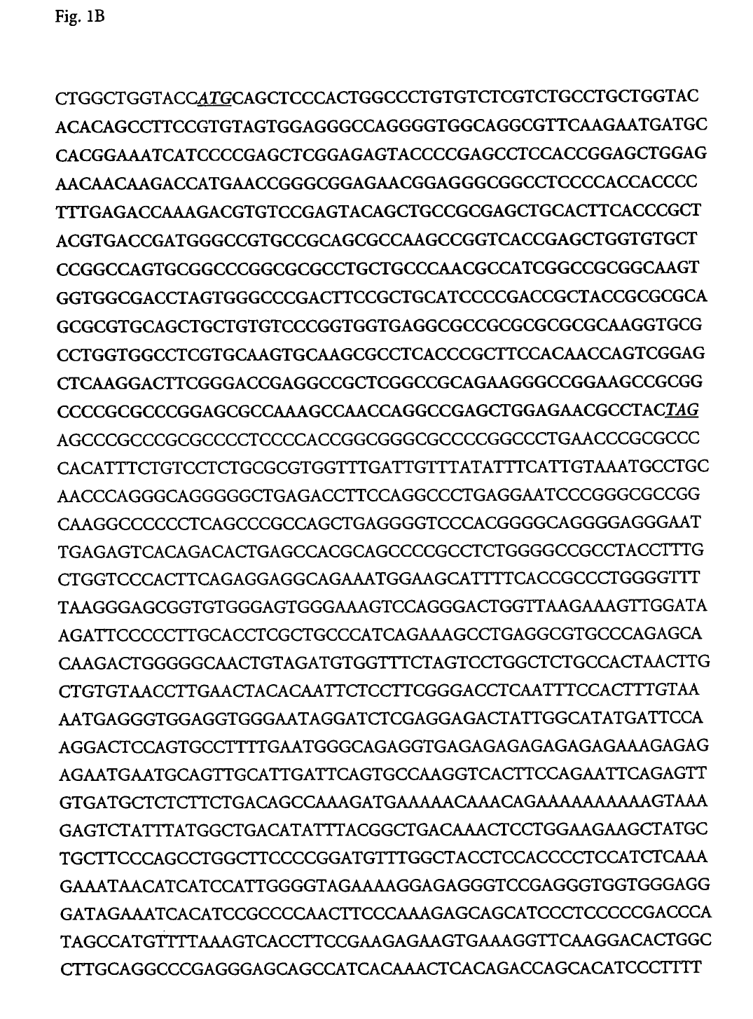

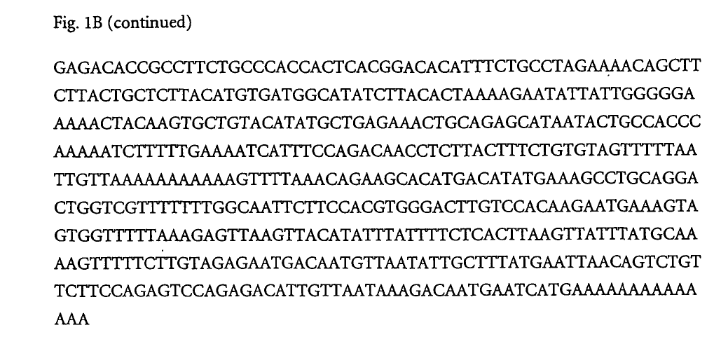

FIG. 1A shows a schematic representation of the genomic organization of the osteolevin gene.

FIG. 1B shows the nucleotide sequence of osteolevin cDNA (SEQ ID NO. 1)

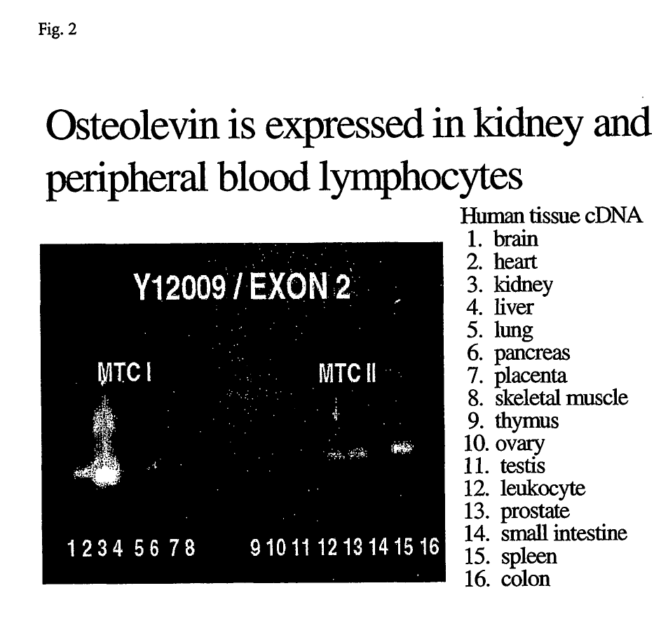

FIG. 2 shows a PCR analysis of osteolevin mRNA expression in various human tissues. The transcript appears widely expressed as demonstrated by observed signals in human cDNA from heart, kidney (strongest expression), pancreas, placenta, prostate, spleen and peripheral blood lymphocytes. Expression was also demonstrated in human osteoblasts.

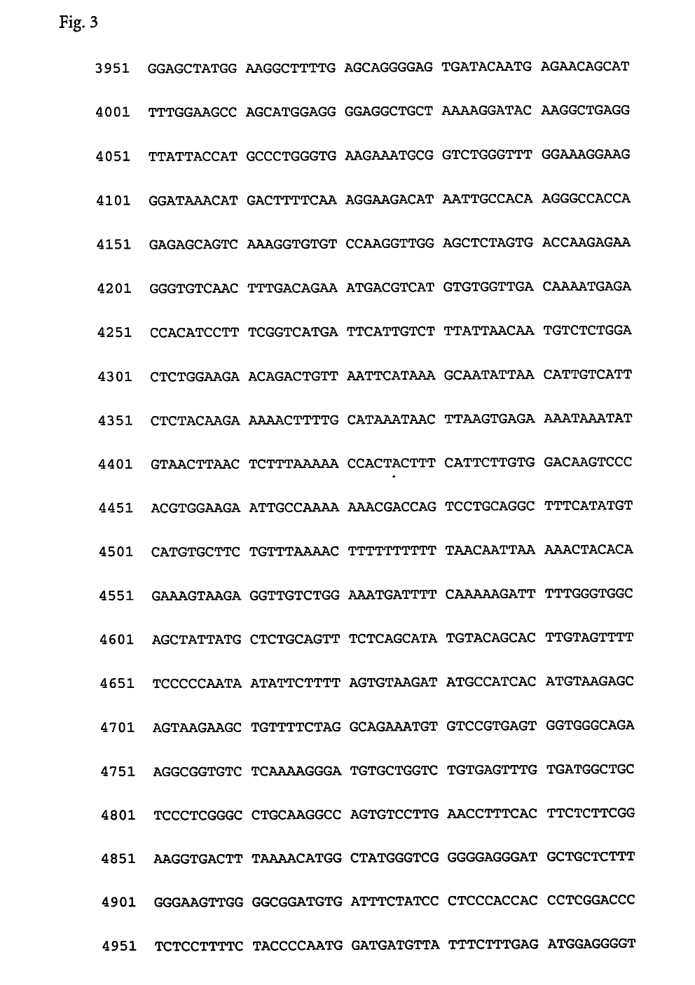

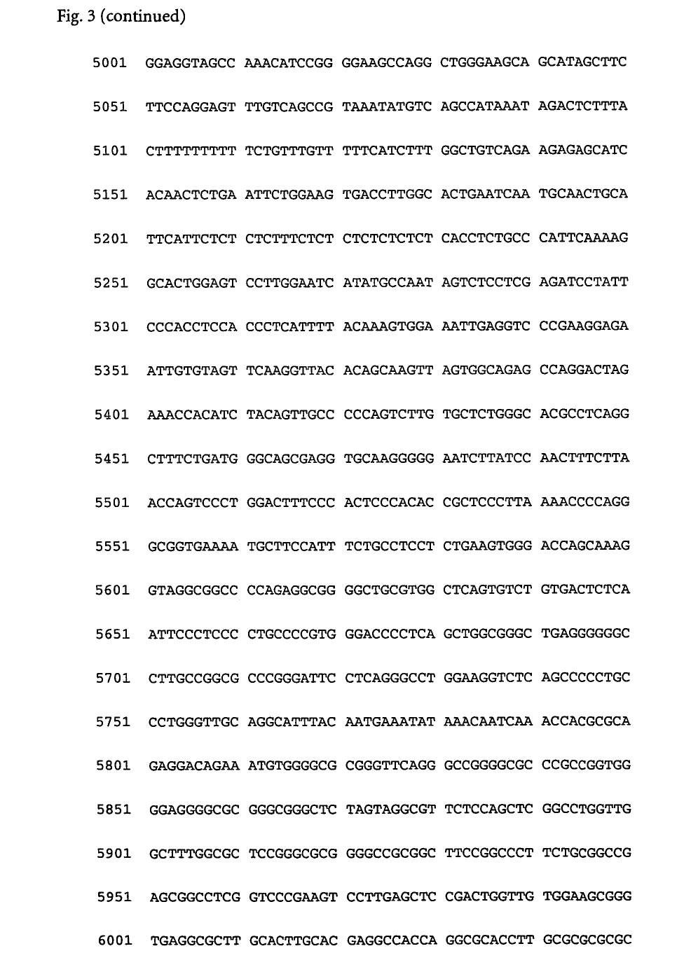

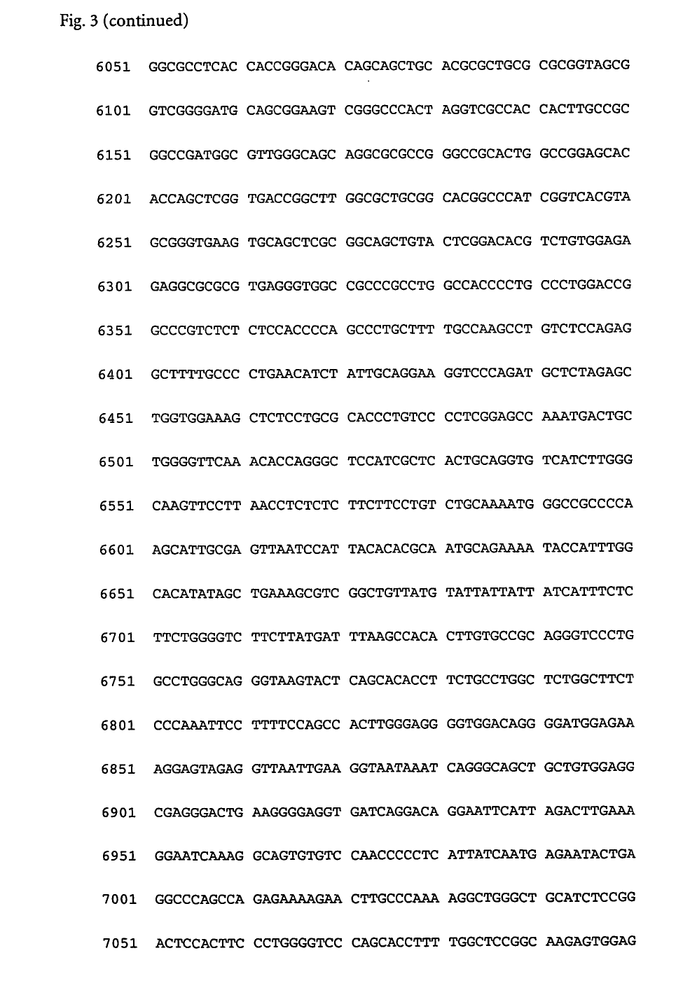

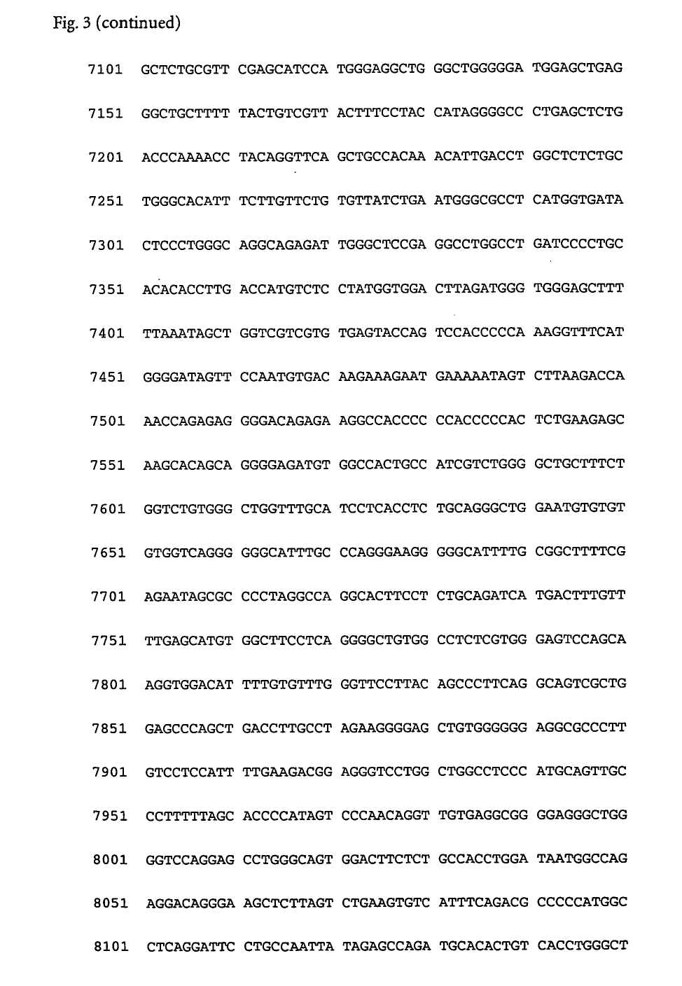

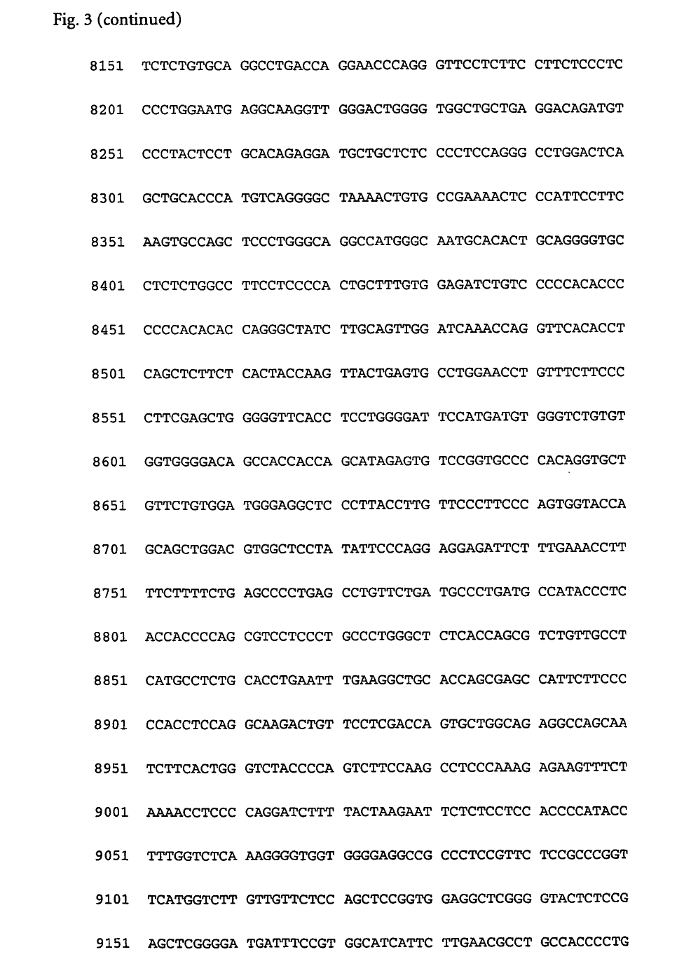

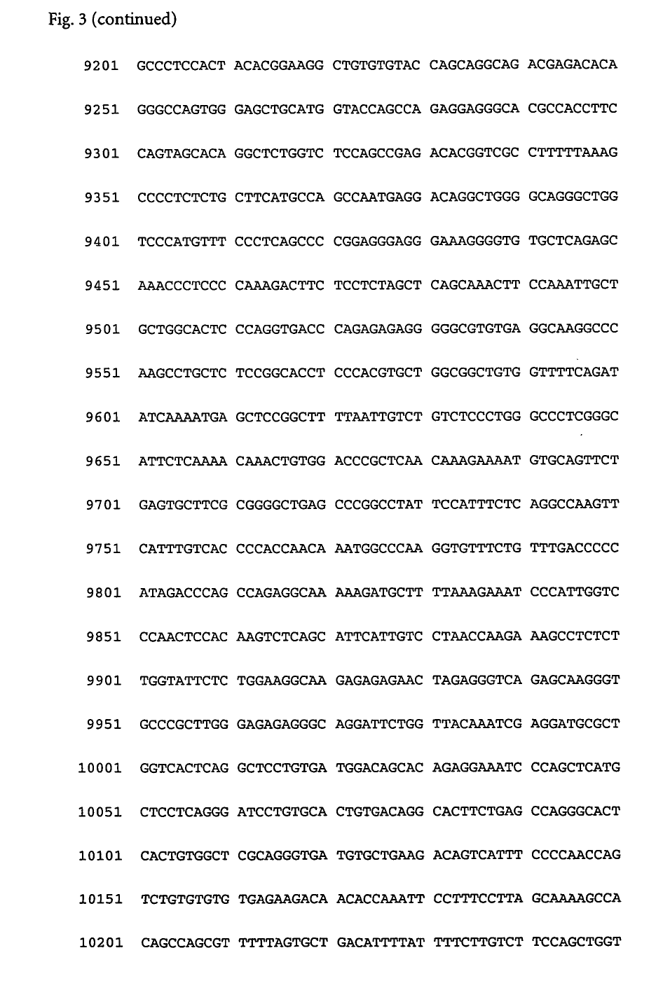

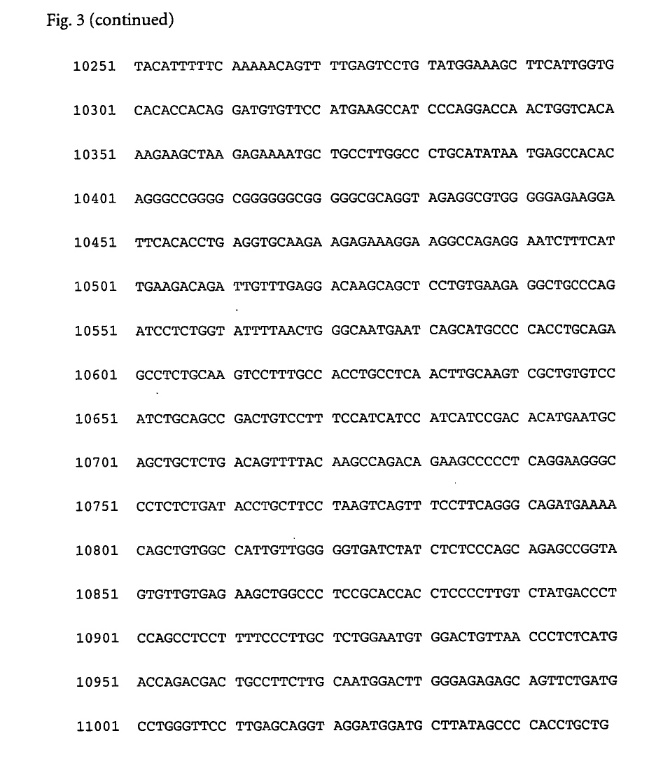

FIG. 3 shows the genomic osteolevin region (SEQ ID NO. 3). Sequence is derived from a HTS large insert clone (EMBL ACCESSION NO. AC003098). Osteolevin sequence is located on the complementary strand. Therefore, reverse complement DNA sequence of genomic osteolevin is shown below.

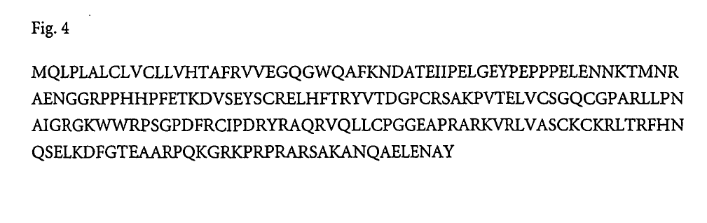

FIG. 4 shows the amino acid sequence of the osteolevin polypeptide (SEQ ID NO. 2).

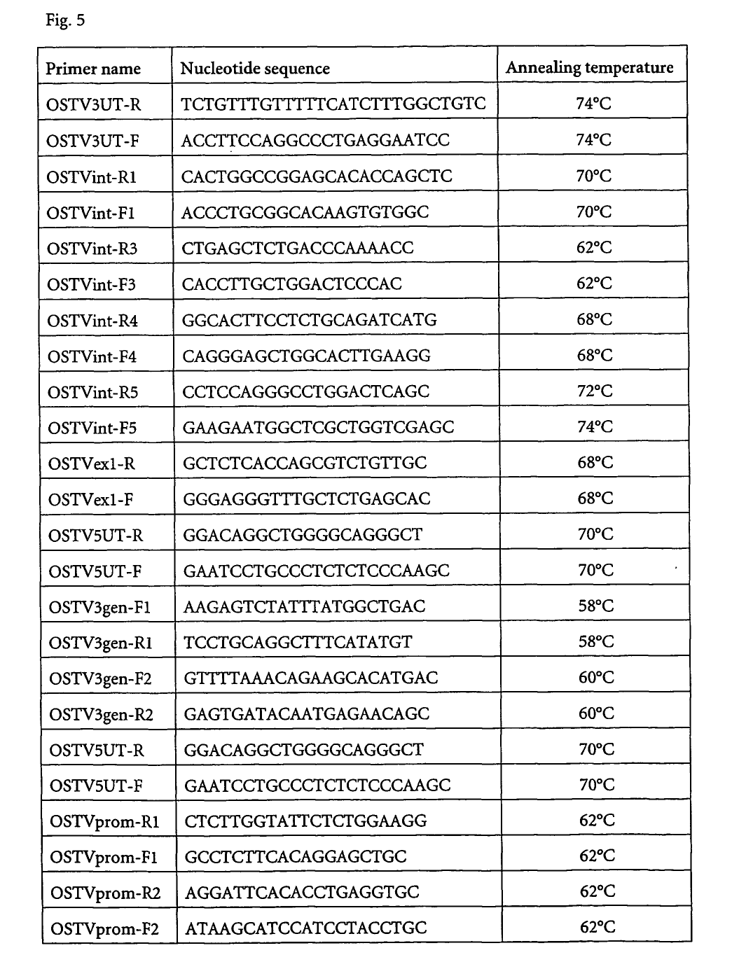

FIG. 5 shows the primers used to amplify the osteolevin regions prior to the screen for polymorphisms by DNA sequencing (SEQ ID No. 4 to 27).

The term “abnormal bone formation” when used herein is broadly defined as net increased or decreased bone formation.

The term “polymorphisms” is broadly defined to include all variations that are known to occur in nudeic and amino acid sequences including insertions, deletions, substitutions and repetitive sequences induding duplications.

The term “osteolevin region” is defined as the area of chromosome 17 which contains the nudeic acid sequences shown in FIG. 3 (SEQ ID NO. 3).

The term “wild-type sequence” when used herein refers to a sequence in the osteolevin region which does not contain polymorphisms.

“Variant” means a variant as defined below having at least about 80% amino acid sequence identity with the deduced osteolevin polypeptide shown in FIG. 4 (SEQ ID NO. 2) including osteolevin nucleotide polymorphisms that occur with a frequency of at least 1% in a certain population. Such variants include polypeptides wherein one or more amino acid residues are added, deleted, or changed anywhere within the sequence, including the N- or C-termini. Ordinarily, an osteolevin variant will have at least about 80% or 85% amino acid sequence identity with the corresponding osteolevin sequence of FIG. 4 (SEQ ID NO. 2), more preferably at least about 90% amino acid sequence identity. Most preferably an osteolevin variant will have at least about 95% amino acid sequence identity with the corresponding osteolevin sequence of FIG. 4 (SEQ ID NO. 2).

“Percent (%) amino acid sequence identity” with respect to the amino acid sequences identified herein is defined as the percentage of amino acid residues in a candidate sequence that are identical with the amino acid residues in the osteolevin sequences, after aligning the sequences in the same reading frame and introducing gaps, if necessary, to achieve the maximum percent sequence identity. Conservative substitutions are not considered to be identical. Alignment for purposes of determining percent amino acid sequence identity can be achieved in various ways that are within the skill in the art, for instance, using publicly available computer software such as BLAST software (see e.g. Altschul et al., J. Mol. Biol., 5; 215(3): 403-410 (1990). Those skilled in the art can determine appropriate parameters for measuring alignment, including any algorithms needed to achieve maximal alignment over the full length of the sequences being compared. “Percent (%) nudeic acid sequence identity” with respect to the osteolevin sequences identified herein is defined as the percentage of nucleotides in a candidate sequence that are identical with the nucleotides in the osteolevin sequences, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST software (see e.g. Altschul et al., J. Mol. Biol., 5; 215(3): 403-410 (1990). Those skilled in the art can determine appropriate parameters for measuring alignment, including any algorithms needed to achieve maximal alignment over the full length of the sequences being compared.

“Isolated”, when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified to a degree sufficient to obtain N-terminal or internal amino acid sequence by use of a spinning cup sequenator, or to homogeneity as assessed by SDS-PAGE under non-reducing or reducing conditions using Coomassie blue or silver stain. Isolated polypeptide includes polypeptide in situ within recombinant cells, since at least one component of the osteolevin natural environment will not be present. Ordinarily, however, isolated polypeptide will be prepared by at least one purification step (referred to herein as an “isolated and purified polypeptide”).

An “isolated” osteolevin nucleic acid molecule is a nucleic acid molecule that is identified and separated from at least one contaminant nucleic acid molecule with which it is ordinarily associated in the natural source of the osteolevin nucleic acid. An isolated osteolevin nucleic acid molecule is other than in the form or setting in which it is found in nature. Isolated osteolevin nucleic acid molecules therefore are distinguished from the osteolevin nucleic acid molecule as it exists in natural cells. However, an isolated osteolevin nucleic acid molecule includes osteolevin nudeic acid molecules contained in cells that ordinarily express osteolevin where, for example, the nucleic acid molecule is in a chromosomal location different from that of natural cells.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking may be accomplished by ligation at convenient restriction sites. If such sites do not exist, the synthetic oligonucleotide adaptors or linkers may be used in accordance with conventional practice.

“Polynucleotide” and “nucleic acid” refer to single or double-stranded molecules which may be DNA, comprised of the nucleotide bases A, T, C and G, or RNA, comprised of the bases A, U (substitutes for T), C, and G. The polynudeotide may represent a coding strand or its complement. Polynucleotide molecules may be identical in sequence to the sequence which is naturally occurring or may include alternative codons which encode the same amino acid as that which is found in the naturally occurring sequence (See, Lewin “Genes V” Oxford University Press Chapter 7, pp. 171-174 (1994). Furthermore, polynucleotide molecules may include codons which represent conservative substitutions of amino acids as described. The polynudeotide may represent genomic DNA or cDNA.

“Polypeptide” refers to a molecule comprised of amino acids which correspond to those encoded by a polynucleotide sequence. The polypeptide may include conservative substitutions where the naturally occurring amino acid is replaced by one having similar properties, where such conservative substitutions do not alter the function of the polypeptide (See, Lewin “Genes V” Oxford University Press Chapter 1, pp.: 9-13 (1994).

The term “antibody” is used in the broadest sense and specifically covers single anti-osteolevin monoclonal antibodies (including agonist, antagonist, and neutralizing antibodies) and anti-osteolevin antibody compositions with polyepitopic specificity. The term “monoclonal antibody” as used herein refers to an antibody obtained from a population of substantially homogeneous antibodies, i.e., the individual antibodies comprising the population are identical except for possible naturally-occurring mutations that may be present in minor amounts.

The present invention relates to a method for screening for osteolevin polymorphism in a subject, said method comprising determining the presence of a polymorphism in the osteolevin nucleic acid sequence obtained from the subject.

Polymorphisms in the osteolevin gene were identified a) by sequencing sclerosteosis patients and comparing the sequence to controls (see Example 2b) and b) by PCR-amplification of 400-600 base pair overlapping fragments (covering the whole genomic region of osteolevin as defined by nucleotide position 4'000-11'000 in EMBL ACCESSION NO. AC003098, Release 62.0) in the DNA of 47 unrelated individuals of 5 different ethnic origin (obtained from Coriell Institute, Hamden, N.J.). Fragments have been sequenced in these 47 samples with a forward and reverse primer, polymorphisms were detected by using the PolyPhred software (licensed from University of Washington) and allele frequencies for the variants were established.

Three different and independent mutations could exclusively be detected in sclerosteosis patients and not in more than 150 control individuals:

A) G to A at position 6136 defined by the position in EMBL ACCESSION NO. AC003098, resulting in a change from Arg(CGA) to a stop codon (TGA). This mutation was found in an homozygous status in two sclerosteosis patients from an American family. Patients were previously described in Balemans et al. (Am. J. Hum. Genet. 64:1661-1669; 1999). One individual from this family, previously identified as disease carrier based on haplotype analysis, was proven to carry the mutation heterozygously.

B) C to T at position 6140 defined by the position in EMBL ACCESSION NO. AC003098, resulting in a change from Trp (TGG) to a stop codon (TGA). This mutation was found in an homozygous status in two sclerosteosis patients from a Brazilian family. Patients were previously described in Balemans et al. (Am. J. Hum. Genet. 64:1661-1669; 1999). Six individuals from this family, previously identified as disease carriers based on haplotype analysis, were proven to carry the mutation heterozygously.

C) T to A at position 9047 defined by the position in EMBL ACCESSION NO.AC003098, resulting in a splice mutation at the begin of intron 1. This mutation was homozygously found in one Italian sclerosteosis patient previously described in Tacconi et al. (Clinical Genetics, 53:497-501; 1998).

Additionally, a number of genetic variants in the osteolevin region have been identified in a standard population panel. 11 base pair substitutions and 1 insertion were detected in 5′ flanking and promoter regions:

| Polymorphism | Position* |
|---|---|
| C to T | 10877 |
| A to G | 10876 |
| T to C | 10817 |
| C to A | 10687 |
| TCC insertion between | 10668 and 10669 |
| C to G | 10424 |
| C to G | 10342 |
| A to G | 10020 |
| T to C | 9783 |
| C to T | 9723 |
| C to T | 9646 |
| G to A | 9616 |

*defined by the position in EMBL ACCESSION NO. AC003098

One amino-acid changing polymorphism was found in exon 1:

| Polymorphism | Position* | Effect |
|---|---|---|
| C to T | 9242 | Changes Valin at position 10 to Isoleucine |

*defined by the position in EMBL ACCESSION NO. AC003098

5 polymorphisms were found in intron 1:

| Polymorphism | Position* |
|---|---|
| A to G | 8375 |
| C to T | 7894 |
| G to T | 7489 |
| T to G | 6358 |

*defined by the position in EMBL ACCESSION NO. AC003098

4 polymorphisms were detected in 3′ flanking region:

| Polymorphism | Position* |
|---|---|
| C to T | 5308 |
| G to A | 5004 |
| C to T | 4866 |
| G to C | 4475 |

*defined by the position in EMBL ACCESSION NO. AC003098

The sequence for EMBL ACCESSION NO. AC003098 is derived from a large insert and is the reverse complement to the 5′ to 3′ osteolevin DNA sequence. Consequently the genetic polymorphisms for the osteolevin cDNA are the reverse complement of the variants described above.

The Van Buchem-sclerosteosis disease interval was previously mapped to an interval of 0.7 cM on human chromosome 17 (Balemans et al., Am. J. Hum. Genet. 64:1661-1669; 1999). Within this interval, a gene encoding osteolevin was identified in silico by analysis of the genomic sequence using GenScan (Burge & Karlin, J. Mol. Biol. 268: 78-94; 1997). With GenScan's default parameter settings, a gene structure with four exons is predicted, of which the two internal ones are assigned lower quality scores than the other two. Homology studies using human protein sequences remotely related to osteolevin revealed that the two internal exons are wrongly predicted by GenScan. Subsequently, comparison to the mouse syntenic region on chromosome 11 (EMBL: AC012296) by dot-plot analysis (“dotter” program: Sonnhammer & Durbin, Gene 167: GC₁₋₁₀; 1995) identified the mouse homologue and unambiguously established the two-exon gene structure.

The genomic organization of the osteolevin gene is illustrated in FIG. 1B. The gene consist of 2 exons, 220 bp and 421 bp in size beginning at bp 9269 and ending at bp 5870 of the sequence shown in FIG. 3. Both exons obey the GT-AG splicing rule. The first in frame ATG occurs in exon 1 (bp 9269) while the TAG stop codon occurs in exon 2 (bp5870) for the putative 213 amino acid protein.

The present invention provides compositions of matter and diagnostic and prognostic methods related to the discovery that polymorphisms in the Van Buchem-scierosteosis disease region may be associated with abnormal bone formation. According to the methods of the present invention, alteration of wild-type osteolevin sequences is detected. “Alteration of wild-type sequences” encompasses all forms of polymorphisms including deletions, insertions and point mutations in the coding and noncoding regions. Polymorphisms may occur anywhere in this region of chromosome 17 including coding and noncoding regions.

A key component of this invention is the delineation of a specific chromosomal region having polymorphisms associated with abnormal bone formation as dearly illustrated by the fact that the above described two nonsense mutations and one splice site mutation in the osteolevin gene all result in sderosteosis, a condition with highly increased bone density as the major hallmark.

The present invention therefore provides methods of identifying novel osteolevin polymorphisms which are correlated with a predisposition for abnormal bone formation by determining one or more sequences in the osteolevin region from individuals known to have abnormal bone formation and then comparing these sequences to that of known osteolevin region wild type sequences.

The presence of the identified osteolevin polymorphisms associated with abnormal bone formation may be ascertained by testing a biological sample from an individual. Biological samples are those samples of materials which have cells containing nucleic acid sequences. Biological samples may be obtained from a variety of sources including blood, tissue, and cell lines. Most simply, blood can be drawn and DNA extracted from the cells of the blood. In addition, prenatal diagnosis can be accomplished by testing fetal cells, placental cells or amniotic cells. Alteration of wild-type osteolevin sequences, whether, for example, by substitution, insertion or deletion, can be detected by any of a variety of means known in the art including the illustrative protocols discussed herein. Further, once a polymorphism is identified, its association with a pathology may be assessed by a variety of statistical and pedigree analyses that are well known in the art. See e.g. Handbook of Human Genetic Linkage (Joseph D. Terwilliger & Jurg Ott eds., 1st ed. 1994); Fundamentals of Biostatistics (Bernard Rosner ed., 1st ed., 1982). For example, evidence that polymorphisms in osteolevin region are associated with abnormal bone formation can be obtained by finding sequences in DNA extracted from affected kindred members that may encode abnormal osteolevin gene products or result in abnormal levels of the gene products. Such abnormal bone formation susceptibility alleles will co-segregate with the disease in large kindreds. They will also be present at a much higher frequency in non-kindred individuals with abnormal bone formation than in individuals in the general population. Moreover, association studies with cases and controls from unrelated individuals may be performed for similar purposes.

The present invention provides isolated nudeic acid molecules comprising a polynucleotide encoding the osteolevin polypeptide having the amino acid sequence shown in FIG. 4 (SEQ ID NO. 2). The nucleotide sequence determined by sequencing the osteolevin cDNA (FIG. 1B; SEQ ID NO. 1) contains an open reading frame encoding a polypeptide of 213 amino acid residues (in bold), including an initiation codon (ATG) at positions 13-15 (cursive type, underlined), with a leader sequence of about 19 amino acid residues, and a predicted molecular weight of about 24 kDa. The stop codon (TAG) appears in cursive type, underlined. The amino acid sequence of the mature osteolevin polypeptide is shown in FIG. 4, amino acid residues 1 to 213 (SEQ ID NO. 2).

Thus, in one embodiment the present invention provides isolated nucleic acid molecules encoding osteolevin selected from the group consisting of a) the nudeic acid molecule or a complement of the nucleic acid molecule set forth in SEQ ID No. 1, b) a nucleic acid molecule or a complement of a nucleic acid molecule encoding a polypeptide set forth in SEQ ID NO. 2, c) a nucleic acid molecule capable of hybridizing to a nucleic acid molecule of above a) or b) and remain stably bound to them under at least moderate, and optionally, under high stringency conditions.

Further embodiments of the present invention include isolated nucleic acid molecules that comprise a polynucleotide having a nucleotide sequence at least 60% identical, preferably at least 80%, 85%, 90% or 95% identical to the nucleic acid molecule set forth in SEQ ID NO. 1.

In another embodiment, the invention provides a recombinant osteolevin polypeptide having an amino acid sequence selected from the group consisting of a) the amino acid sequence encoded by anyone of the above nucleic acid molecules, b) an amino acid sequence which is at least 80% identical, preferably at least 90% identical, still more preferably at least 95% identical with the amino acid sequence set forth in SEQ ID NO. 2, c) the amino acid sequence set forth in SEQ ID NO. 2.

The polypeptides of the present invention also include polypeptides having an amino acid sequence with at least 90% similarity, preferably at least 95% similarity to the amino acid sequence set forth in SEQ ID NO. 2, as well as polypeptides having an amino acid sequence at least 60% identical, preferably at least 80%, 85%, 90% or 95%, more preferably at least 96%, 97%, 98% or 99% identical to the amino acid sequence set forth in SEQ ID NO. 2.

Furthermore, the present invention provides isolated nucleotide sequences from the osteolevin region having polymorphisms that may be associated with abnormal bone formation. The invention further provides variations and modifications of these sequences and molecules that they encode using methods that are well known in the art such as site-directed PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34.-315 (1985)), restriction selection mutagenesis (Wells et al., Philos. Trans. R. Soc. London SerA, 317:415 (1986)) or other known techniques can be performed on the cloned DNA to produce the variant DNA. Covalent modifications of the sequences disclosed herein are included within the scope of this invention. See, e.g., Current Protocols In Molecular Biology, Volume 2, Units 10, 11 and 14, Frederick M. Ausubel et al. eds., 1995; Molecular Cloning, A Laboratory Manual, §12, Tom Maniatis et al. eds., 2nd ed. 1989.

The description below relates primarily to production of the sequences of the invention by culturing cells transformed or transfected with a vector containing polymorphic sequences of the osteolevin region. It is contemplated that alternative methods, which are well known in the art, may be employed to prepare these molecules. For instance polymorphic sequences or portions thereof may be produced by direct oligomer or peptide synthesis using solid-phase techniques (see, e.g., Stewart et al., Solid-Phase Peptide Synthesis, W.H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) and following the manufacturer's instructions. Various portions of the osteolevin sequences may be chemically synthesized separately and combined using chemical or enzymatic methods.

DNA having polymorphic sequences of the present invention may be obtained from genomic or cDNA libraries prepared from tissue from individuals having these sequences or by oligonucleotide synthesis as outlined above. Libraries can be screened with probes (such as oligonucleotides of at least about 20-80 bases) designed to identify the sequence of interest or the protein encoded by it. Illustrative libraries include λTriplEx human kidney cDNA library (Clontech Laboratories, Inc.) and λTriplEx human spleen cDNA library (Clontech Laboratories, Inc.). Screening the cDNA or genomic library with the selected probe may be conducted using standard procedures, such as described in Sambrook et al., Molecular Cloning. A Laboratory Manual (New York: Cold Spring Harbor Laboratory Press, 1989). An alternative means to isolate a gene encoded by the osteolevin region is to use PCR methodology [Sambrook et al., supra; Dieffenbach et al., PCR Primer: A Laboratory Manual (Cold Spring Harbor Laboratory Press, 1995)].

The Examples below describe techniques for screening DNA libraries. The oligonudeotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²p-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs that employ various algorithms to measure homology.

Nucleic acid having protein coding sequences may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

Large amounts of the polynucleotides of the present invention may be produced by replication in a suitable host cell using any of a variety of techniques that are well known in the art. Natural or synthetic polynucleotide fragments coding for a desired fragment will be incorporated into recombinant polynucleotide constructs, usually DNA constructs, capable of introduction into and replication in a prokaryotic or eukaryotic cell. Usually the polynucleotide constructs will be suitable for replication in a unicellular host, such as yeast or bacteria, but may also be intended for introduction to (with and without integration within the genome) cultured mammalian or plant or other eukaryotic cell lines. The purification of nudeic acids produced by the methods of the present invention are described, e.g., in Sambrook et al., 1989 or Ausubel et al., 1992.

Polynucleotide constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Such vectors may be prepared by means of standard recombinant techniques well known in the art and discussed, for example, in Sambrook et al., 1989 or Ausubel et al., 1992. An appropriate promoter and other necessary vector sequences will be selected so as to be functional in the host and may include, when appropriate, those naturally associated with the osteolevin region. Examples of workable combinations of cell lines and expression vectors are described in Sambrook et al., 1989 or Ausubel et al., 1992. Many useful vectors are known in the art and may be obtained from such vendors as Stratagene, New England Biolabs, Promega Biotech, Invitrogen, Pharmingen and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Vectors and promoters suitable for use in yeast expression are further described in Hitzeman et al., EP 73,675A. Appropriate non-native mammalian promoters might include the early and late promoters from SV40 or promoters derived from murine Molony leukemia virus, mouse tumor virus, avian sarcoma viruses, adenovirus II, bovine papilloma virus or polyoma. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, Cold Spring Harbor, N.Y. (1983). Sequences encoding strong secretion signal peptides may also be operably linked to the sequence of the mature osteolevin protein, in which the osteolevin signal peptide sequence has been eliminated. For example, the signal sequence for gp67 may be included in a baculoviral vector to promote the secretion of the polypeptide from insect cells. Such vectors are commercially available from Pharmingen and other sources. While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells which express the inserts. Typical selection genes encode proteins that a) confer resistance to antibiotics or other toxic substances, e.g. ampicillin, neomycin, methotrexate, etc., b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection, or the vectors can be introduced directly into host cells by methods well known in the art, which vary depending on the type of cellular host, including electroporation; transfection employing calcium chloride, rubidium chloride calcium phosphate, DEAE-dextran, or other substances; microprojectile bombardment; lipofection; infection (where the vector is an infectious agent, such as a retroviral genome); and other methods. See generally, Sambrook et al., 1989 and Ausubel et al., 1992. The introduction of the polynucleotides into the host cell by any method known in the art, including, inter alia, those described above, will be referred to herein as “transformation.” The cells into which have been introduced nucleic acids described above are meant to also include the progeny of such cells.

Large quantities of the nucleic acids and polypeptides of the present invention may be prepared by expressing osteolevin nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used. Mammalian or other eukaryotic host cells, such as those of yeast, filamentous fungi, plant, insect, or amphibian or avian species, may also be useful for production of the proteins of the present invention. Propagation of mammalian cells in culture is per se well known. Examples of commonly used mammalian host cell lines are VERO and HeLa cells, Chinese hamster ovary (CHO) cells, and W138, BHK, and COS cell lines, although it will be appreciated by the skilled practitioner that other cell lines may be appropriate, e.g., to provide higher expression, desirable glycosylation patterns, or other features.

Clones are selected by using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

Prokaryotic or eukaryotic cells transformed with the polynucleotides of the present invention will be useful not only for the production of the nucleic acids and polypeptides of the present invention, but also, for example, in studying the characteristics of osteolevin polypeptides. The probes and primers based on sequences disclosed herein can be used to identify homologous sequences and proteins in other species such as the murine osteolevin gene. Furthermore, homologous sequences from other species can be identified in silico by querying databases of such species' genomic or cDNA sequence with human osteolevin sequence. The species-specific done can then be obtained using polymerase chain reaction (PCR). These gene sequences and proteins are used in the diagnostic/prognostic, therapeutic and drug screening methods described herein for the species from which they have been isolated.

Nucleic acid sequences having polymorphisms associated with abnormal bone formation can be detected by hybridization with a polynucleotide probe which forms a stable hybrid with that of the target sequence, under stringent to moderately stringent hybridization and wash conditions. The present invention allows for the design of probes which preferentially hybridize to polymorphic regions. The design of probes which preferentially target specific sequences and hybridization conditions for their use is well known in the art. See e.g. Current Protocols In Molecular Biology, Volumes I-III, Frederick M. Ausubel et al. eds., 1995. For example, if it is expected that the probes will be perfectly complementary to the target sequence, stringent conditions will be used. Hybridization stringency may be lessened if some mismatching is expected, for example, if variants are expected with the result that the probe will not be completely complementary. Conditions are chosen which rule out nonspecific/adventitious bindings in order to minimize noise.

Probes for polymorphisms in the osteolevin region may be of any suitable length, which are proximal to or span all or a portion of the polymorphism, and which allow preferential hybridization to the region. If the target sequence contains a sequence identical to that of the probe, the probes may be short, e.g., in the range of about 8-30 base pairs, since the hybrid will be relatively stable under even stringent conditions. If some degree of mismatch is expected with the probe, i.e., if it is suspected that the probe will hybridize to a variant region, a longer probe may be employed which hybridizes to the target sequence with the requisite specificity.

The probes can include an isolated polynucleotide attached to a label or reporter molecule and may be used to isolate other polynucleotide sequences, having sequence similarity or being proximal to the sequences of interest by standard methods. For techniques for preparing and labeling probes see, e.g., Sambrook et al., 1989 or Ausubel et al., 1992. Other similar polynucleotides may be selected by using homologous polynucleotides. Alternatively, polynucleotides encoding these or similar polypeptides may be synthesized or selected by use of the redundancy in the genetic code. In expressed sequences, various codon substitutions may be introduced, e.g., by silent changes (thereby producing various restriction sites) or to optimize expression for a particular system. Polymorphisms may be introduced to modify the properties of the polypeptide, perhaps to change the polypeptide degradation or turnover rate. Probes comprising synthetic oligonucleotides or other polynucleotides of the present invention may be derived from naturally occurring or recombinant single- or double-stranded polynudeotides, or be chemically synthesized. Probes may also be labeled by nick translation, Kienow fill-in reaction, or other methods known in the art.

The design of probes with the appropriate size and sequence for preferential binding to target specific sequences as well as hybridization conditions for their use is well known in the art. See, e.g., Current Protocols In Molecular Biology, Volumes 1, units 2, 4, and 6, Frederick M. Ausubel et al. eds., 1995. Portions of polynucleotide sequences having at least about eight nudeotides, usually at least about 15 nucleotides, and fewer than about 6 kb, usually fewer than about 1.0 kb, from a polymorphic sequence are preferred as probes. Also contemplated are probes having a specific portion of a polymorphic sequence. Moreover, probes which are proximal to a polymorphic region may also be used in evaluating nucleic acid samples. In addition to their use in evaluating genomic sequences, the probes may also be used to determine whether mRNA such as that encoding osteolevin is present in a cell or tissue.

In order to detect the presence of a polymorphism associated with abnormal bone formation, a biological sample such as blood is prepared and analyzed for the presence or absence of polymorphic sequences. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis.

The identification of the association between polymorphisms in the osteolevin region and abnormal bone formation permits the early presymptomatic screening of individuals to identify those at risk for having pathologies associated with abnormal bone formation. To identify such individuals, the osteolevin region is screened for polymorphisms either directly or after cloning the sequences of interest. There are a number of different methods of the invention which may be employed both to evaluate individuals for potentially pathogenic polymorphisms and to specifically characterize those polymorphisms which are associated with abnormal bone formation. For example, the invention provides a method for screening for a polymorphism associated with abnormal bone formation in an individual. Moreover, the invention also provides a method of identifying a polymorphism associated with abnormal bone formation by comparing a osteolevin sequence isolated from an affected subject to a known wild type osteolevin sequence and identifying recurrent polymorphisms that are associated with abnormal bone formation.

As discussed below, samples can be tested for the presence of nucleic acid sequences which are different from normal sequences using any one of a wide variety of differential nucleic acid analysis techniques that are well known in the art. Differential nucleic acid analysis techniques include, but are not limited to: fluorescent in situ hybridization (FISH), direct DNA sequencing, single stranded conformational analysis (SSCP), Southern blotting including restriction fragment length polymorphism analysis (RFLP), the polymerase chain reaction (PCR), polymorphism specific oligonucleotide hybridizations and PCR-SSCP analysis. As discussed below, for sequences coding for expressed molecules and polypeptides, additional techniques may also be utilized. For a review of techniques for evaluating and manipulating nucleic and amino acid sequences, see Current Protocols In Molecular Biology, Volumes I-III, Frederick M. Ausubel et al. eds., 1995.

Alteration of osteolevin mRNA expression can be detected by any techniques known in the art. These include Northern blot analysis, quantitative PCR amplification (TaqMan), and RNase protection. Diminished mRNA expression indicates an alteration of the wild-type gene locus. Alteration of wild-type genes can also be detected by screening for alteration of wild-type osteolevin protein. For example, monoclonal antibodies immunoreactive with specific osteolevin epitopes can be used to screen a tissue. Lack of cognate antigen would indicate a polymorphism. Antibodies specific for products of mutant alleles could also be used to detect mutant gene product. Such immunological assays can be done in any convenient formats known in the art. These include Western blots, immunohistochemical assays and ELISA assays. Any means for detecting an altered protein can be used to detect alteration of wild-type osteolevin. Functional assays, such as protein binding determinations, can be used. In addition, assays can be used which detect the biochemical function of genes in the osteolevin region. Typically, finding an alteration in the biochemical function of a polypeptide encoded by a gene in this region can indicate alteration of a wild-type gene in this region.

Protein localization at the cellular level and subcellular levels can be determined by epitope tagging, a method that utilizes antibodies against guest peptides. Epitope tagging begins with a cloned gene and an antibody that recognizes a known peptide (the epitope). Using recombinant DNA technology, a sequence of nucleotides encoding the epitope is inserted into the coding region of the cloned gene, and the hybrid gene is introduced into a cell by a method such as transformation. When the hybrid gene is expressed the result is a chimeric protein containing the epitope as a guest peptide. If the epitope is exposed on the surface of the protein, it is available for recognition by the epitope-specific antibody, allowing the investigator to observe the protein within the cell using immunofluorescence or other immunolocalization techniques. Therefore, operably linked sequences which encode peptide tags may be included in the vector. The peptide tags are typically 8-12 amino acids but may be longer. The tags can be recognized by specific antibodies (e.g. the myc epitope) or may bind particular metals (e.g. His6 tag binds nickel columns). The tags facilitate the purification of the osteolevin protein from its source.

A number of methods can be used to directly detect DNA sequence variation. Direct DNA sequencing, either manual sequencing or automated fluorescent sequencing can detect sequence variation. The allele(s) of genes in the osteolevin region in an individual to be tested can be cloned using conventional techniques. For example, a blood sample is obtained from the individual, osteolevin genomic DNA is isolated from the cells in this sample and ligated into an appropriate vector for amplification. The sequences of the clones can then be determined and compared to the normal osteolevin sequences. Techniques involving DNA cloning and sequencing are well known in the art, see e.g. Current Protocols In Molecular Biology, Volume 1, unit 7, Frederick M. Ausubul et al. eds., 1995.

Another approach to detect variations in DNA sequences is the single-stranded conformation polymorphism assay (SSCP) (Orita et al., 1989). This method does not detect all sequence changes, especially if the DNA fragment size is greater than 200 bp, but can be optimized to detect most DNA sequence variation. The reduced detection sensitivity is a disadvantage, but the increased throughput possible with SSCP makes it an attractive, viable alternative to direct sequencing for polymorphism detection on a research basis. The fragments which have shifted mobility on SSCP gels are sequenced to determine the exact nature of the DNA sequence variation. Other approaches based on the detection of mismatches between the two complementary DNA strands include clamped denaturing gel electrophoresis (CDGE) (Sheffield et al., Am. J. Hum. Genet., 49: 699-706 (1991)), heteroduplex analysis (NA) Mite et al., Genomics 12: 301-306 (1992)) and chemical mismatch cleavage (CMC) (Grompe et al., P.N.A.S. 86: 5855-5892 (1989)). Other methods which might detect these classes of polymorphisms such as a protein truncation assay or the asymmetric assay, detect only specific types of polymorphisms and would not detect missense polymorphisms. A review of currently available methods of detecting DNA sequence variation can be found in a recent review by Grompe et al., Nature Genetics 5: 111-117, (1993) and Landegren et al, Genome Research8:769-776, (1998).

A rapid preliminary analysis to detect polymorphisms in DNA sequences can be performed using RFLP, where DNA is cut with one or more restriction enzymes, preferably with a large number of restriction enzymes and analyzed with osteolevin specific probes in a series of Southern blots. Each blot contains a series of normal individuals and a series of cases with abnormal bone formation. Southern blots displaying hybridizing fragments (differing in length from control DNA when probed with sequences near or including known polymorphic loci) indicate a possible polymorphism. Techniques involving RFLP are well known in the art, see, e.g., Current Protocols In Molecular Biology, Volume 1, unit 2, Frederick M. Ausubul et al. eds., 1995.

Restriction fragment length polymorphism analysis is a preferred method of analysis due to its ability to identify uncharacterized polymorphisms. Specifically, by simply using sequences from various regions in osteolevin as probes, the skilled practitioner may evaluate nudeic acid samples for a wide variety of polymorphisms including those which have yet to be identified. Probes in these analyses may include sequences having the illustrative polymorphisms (e.g. such as the T/C promoter polymorphism at position 10877 in the EMBL ACCESSION NO. AC003098) disclosed herein or alternatively, may include proximal sequences identified herein or isolated by chromosomal walking techniques that are well known in the art. See e.g. Ueghara et al., Mamm Genome 1(2): 92-99 (1991).

A particularly preferred method of nucleic acid analysis using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be analyzed by restriction endonudease digestion, sequenced or used as a substrate for DNA probes. When the sequences encompassing a specific polymorphism are known, a variety of PCR primers targeting these sequences may be generated. For example, sequences flanking the polymorphism may be used to amplify those sequences. For a variation of sequence-specific PCR, primers can be used which hybridize at their 3′ ends to a particular osteolevin polymorphism. If the particular polymorphism is not present, an amplification product is not observed. Amplification Refractory Polymorphism System (ARMS) can also be used, as disclosed in European Patent Application Publication No. 0332435 and in Newton et al., 1989. Alternatively, polymerase chain reactions (PCRs) can be performed with primer pairs for the 5′ region or the exons of the osteolevin gene. PCRs can also be performed with primer pairs based on any sequence of the normal osteolevin region. For example, primer pairs for one of the introns can be prepared and utilized. Finally, PCR can also be performed on the mRNA, following conversion of the mRNA to cDNA by reverse transcription. The amplified products are then analyzed by single stranded conformation polymorphisms (SSCP) using conventional techniques to identify any differences and these are then sequenced and compared to the normal gene sequence.

Primer pairs of the present invention are useful for determination of the nudeotide sequence of a particular osteolevin sequence using PCR. For example, the pairs of single-stranded DNA primers can be annealed to sequences within or surrounding osteolevin sequences on chromosome 17 in order to prime amplifying DNA synthesis of the gene itself. A complete set of these primers allows synthesis of all of the nucleotides of the gene coding sequences, i.e., the exons. The set of primers preferably allows synthesis of both intron and exon sequences. In addition, allele-specific primers can also be used. Such primers anneal only to particular osteolevin mutant alleles, and thus will only amplify a product in the presence of the mutant allele as a template.

In order to facilitate subsequent cloning of amplified sequences, primers may have restriction enzyme site sequences appended to their 5′ ends. For example, all nudeotides of the primers can be derived from sequences adjacent to one or more osteolevin polymorphisms, except for the few nucleotides necessary to form a restriction enzyme site. Such enzymes and sites are well known in the art. The primers themselves can be synthesized using techniques which are well known in the art. Generally, the primers can be made using oligonudeotide synthesizing machines which are commercially available. Given the level of skill in the art, the design of particular primers is well within the skill of the art. See, e.g., Current Protocols In Molecular Biology, Volume II, unit 15, Frederick M. Ausubel et al. eds., 1995.

DNA sequences of the osteolevin region which have been amplified by use of PCR may also be screened using allele-specific probes. These probes are nucleic acid oligomers, each of which contains a region of the gene sequence harboring a known polymorphism. For example, one oligomer may be about 20 nucleotides in length, corresponding to a portion of the osteolevin polymorphic sequence. By use of a battery of such allele-specific probes, PCR amplification products can be screened to identify the presence of a previously identified polymorphism in the gene. Hybridization of allele-specific probes with amplified osteolevin sequences can be performed, for example, on a nylon filter. Hybridization to a particular probe under stringent hybridization conditions indicates the presence of the same polymorphism in the tissue as in the allele-specific probe. Individuals can be quickly screened for common osteolevin variants by amplifying the individual's DNA using suitable primer pairs and analyzing the amplified product, e.g., by dot-blot hybridization using allele-specific oligonucleotide probes. Once a polymorphism has been characterized, an allele specific detection approach such as allele specific oligonucleotide (ASO) hybridization can be utilized to rapidly screen large numbers of samples.

Another preferentially applied method to detect polymorphisms include the use of mass spectrometry. After the PCR amplification of a DNA sequence that contains a polymorphism as e.g. T/ C at position 9783 defined by EMBL ACCESSION NO. AC003098, an internal primer extension reaction is carried out with a primer ending one base upstream from the polymorphism of interest. Using only dideoxynucleoside triphosphates (ddNTPs) in the primer extension reaction the primer will be extended by only one base which represents the polymorphic site (e.g. position 9783). The exact mass of the extended primer is determined directly with MALDI-TOF (Matrix Assisted Laser Desorption Ionization—Time of Flight) mass spectrometry and heterozygotes generate 2 peaks that can be unambiguously distinguished.

Invasive cleavage products may also be detected by mass spectrometry or by fluorescent based methods. Single nucleotide polymorphisms (SNPs) are detected based on the ability of special structure-specific endonucleases (cleavases) to recognize specific DNA structures (created by a specific hybridisation). An invader probe and a labeled signal probe are designed to hybridize to the target DNA so that the Invader probe overlaps the signal probe by at least one base representing the SNP site. This invasion of the signal-probe target duplex displaces a single-stranded flap containing the label. The juncture between the flap and the partially invaded duplex is recognized and cleaved by the enzyme only in case of complementary bases at the cleavage site, releasing the unhybridized region of the signal probe. Detection of the cleaved fragment can be accomplished as described above or by direct gel analysis or enzyme-linked antibody to a tag on the fragment. After cleavage, a new signal probe hybridizes and the process repeats, so that the cleaved signal probe accumulates. The signal is therefore amplified in this method and this amplification increases the overall sensitivity of the technique.

Vice versa, several polymorphism-containing oligonucleotides may be immobilized on a nylon filter (“SNP strip”) and hybridized with the products of a multiplex PCR reaction obtained from the DNA of an individual for allele-specific hybridisation (Cheng et al., Clin. Chem. Lab. Med. (1998) 36(8): 561-566, RMS, Alameda).

The majority of the diagnostic assays described above incorporate nudeic acid probes as a crucial element. When the probes are used to detect the presence of the target sequences, the biological sample to be analyzed, such as blood or serum, may be treated to extract the nucleic acids. As discussed above, the sample nucleic acid may be prepared in various ways to facilitate detection of the target sequence, e.g., denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques known in the art.

Target nucleic acids, probe and analyte can be incubated under conditions which promote stable hybrid formation of the target sequence in the probe with the putative targeted sequence in the analyte. The region of the probe which is used to bind to the analyte can be made completely complementary to the targeted region of human chromosome 17. Therefore, high stringency conditions are desirable in order to prevent false positives. However, conditions of high stringency are used only if the probes are complementary to regions of the chromosome which are unique in the genome. The stringency of hybridization is determined by a number of factors during hybridization and during the washing procedure, including temperature, ionic strength, base composition, probe length, and concentration of formamide. These factors are outlined in, for example, Maniatis et al., Molecular Cloning, A Laboratory Manual, Cold Springs Harbor Laboratory, 1982 and Sambrook et al., 1989. Under certain circumstances, the formation of higher order hybrids, such triplexes, quadraplexes, etc., may be desired to provide the means of detecting target sequences.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. Probe sequences may also hybridize specifically to duplex DNA under certain conditions to form triplex or other higher order DNA complexes. The preparation of such probes and suitable hybridization conditions are well known in the art.

Detection, if any, of the resulting hybrid is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal front the labeled moiety. A number of these variations are reviewed in, e.g., Matthews & Kricka, Anal. Biochem., 169: 1, 1988; Landegren et al., Science, 242: 229, 1988; Mittlin, 1989; U.S. Pat. No. 4,868,105; and in EPO Publication No. 225,807.

As noted above, a number of non-PCR based screening assays are contemplated in this invention. One procedure hybridizes a nucleic acid probe (or an analog such as a methyl phosphonate backbone replacing the normal phosphodiester), to the DNA target present at a low concentration. This probe may have an enzyme covalently linked to the probe, such that the covalent linkage does not interfere with the specificity of the hybridization. This enzyme-probe-conjugate target nucleic acid complex can then be isolated away from the free probe enzyme conjugate and a substrate is added for enzyme detection. Enzymatic activity is observed as a change in color development or luminescent output resulting in an increase in sensitivity. For an example relating to the preparation of oligodeoxynucleotide-alkaline phosphatase conjugates and their use as hybridization probes, see Jablonski et al., N.A.R., 14: 6115-6128, 1986. Two-step label amplification methodologies are known in the art. These assays work on the principle that a small ligand (such as digoxigenin, biotin, or the like) is attached to a nudeic acid probe capable of specifically binding an osteolevin region sequence. Allele specific probes are also contemplated within the scope of this example and exemplary allele specific probes include probes encompassing the predisposing polymorphisms of this patent application.

In one example, the small ligand attached to the nucleic acid probe is specifically recognized by an antibody-enzyme conjugate. In one embodiment of this example, digoxigenin is attached to the nudeic acid probe. Hybridization is detected by an anti-digoxigenin antibody conjugated to alkaline phosphatase conjugate. The alkaline phosphatase modifies a chemiluminescent substrate which can then be detected. For methods for labeling nucleic acid probes according to this embodiment see Martin et al., BioTechniques 9: 762-768, 1990. In a second example, the small ligand is recognized by a second ligand-enzyme conjugate that is capable of specifically complexing to the first ligand. A well known embodiment of this example is the biotin-avidin type of interaction. For methods for labeling nudeic acid probes and their use in biotin-avidin based assays see Nguyen et al., BioTechniques 13: 116-123, 1992. It is also contemplated within the scope of this invention that the nucleic acid probe assays of this invention will employ a combination of nucleic acid probes capable of detecting osteolevin polymorphisms. Thus, in one example to detect the presence of polymorphisms in a cell sample, more than one probe complementary to the gene is employed and in particular the number of different probes is alternatively two, three, or five different nucleic acid probe sequences. In another example, to detect the presence of polymorphisms in the osteolevin region sequence in a patient, more than one probe complementary to the genes in the region is employed. The cocktail includes probes capable of binding to the allele-specific polymorphisms identified in populations of patients with alterations in this region. In this embodiment, any number of probes can be used, and will preferably include probes corresponding to the major polymorphisms in the Van Buchem-sclerosteosis disease region identified as being associated with abnormal bone formation.

Any sequence differences found by one of the techniques discussed above will identify an individual as having a molecular variant of the osteolevin region that may associate with abnormal bone formation. These variants can take a number of forms and can occur in both coding and non-coding regions. Certain polymorphisms associated with an expressed gene could generate an abnormal protein or could significantly alter protein expression. Additional disruptive polymorphisms could include small in-frame deletions and non-conservative base pair substitutions which could have a significant effect on the protein produced, such as changes to or from a cysteine residue, from a basic to an acidic amino acid or vice versa, from a hydrophobic to hydrophilic amino acid or vice versa, or other polymorphisms which would affect secondary or tertiary protein structure. Silent polymorphisms or those resulting in conservative amino acid substitutions would not generally be expected to disrupt protein function.

The methods and osteolevin sequences disclosed herein also provide for a variety of assays using DNA chip technology (see e.g. Wang et al., Science 15; 280: 1077-1082 (1998) and U.S. Pat. Nos. 5,858,661 and 5,837,832 which are incorporated herein by reference). In particular, the present invention provides arrays of osteolevin specific oligonucleotide probes immobilized on a solid support (or “chip”). In this context, DNA chips containing arrays of oligonucleotide probes can be used to determine whether a target nucleic acid sample contains a nudeotide sequence identical to, or different from, a specific reference sequence. An exemplary array comprises probes exactly complementary to the reference sequence (such as the TCC insertion at position 10668 defined by EMBL ACCESSION NO. AC003098 in the osteolevin sequence), as well as probes that differ by one or more bases from the exactly complementary probes. In a typical embodiment, an array will comprise a set of oligonucleotide probes such that, for each base in a specific reference sequence, the set includes a probe that is exactly complementary to a section of the reference osteolevin sequence and additional probes which are related to this reference sequence except that one or more nucleotides within this sequence been replaced by a predetermined set of nudeotides (typically encompassing a portion of a polymorphic region). The detection of sequences binding to such arrays can be carried out by a variety of methods that are known in the art (see e.g. U.S. Pat. No. 5,837,832).

The present invention further provides antibodies to polymorphic regions of proteins encoded by genes in the osteolevin region. Exemplary antibodies include polyclonal, monospecific polyclonal, monoclonal, humanized, bispecific, and heteroconjugate antibodies well known in the art. In particular, the presence of abnormal bone formation can also be detected on the basis of the alteration of wild-type osteolevin polypeptide. While such alterations can be determined by sequence analysis in accordance with conventional techniques, more preferably, antibodies (polyclonal or monoclonal) are used to detect differences in, or the absence of osteolevin peptides. Techniques for generating and purifying antibodies are well known in the art and any such techniques may be chosen to achieve the preparations claimed in this invention. In a preferred embodiment of the invention, antibodies will immunoprecipitate polymorphic osteolevin proteins from solution as well as react with these proteins on Western or immunoblots of polyacrylamide gels. In another preferred embodiment, antibodies will detect osteolevin proteins in paraffin or frozen tissue sections, using immunocytochemical techniques.

Preferred embodiments relating to methods for detecting osteolevin polypeptides or their polymorphisms include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference.

Nucleic acids which encode genes in the osteolevin region or their modified forms can also be used to generate either transgenic animals or “knock out” animals which are useful in the development and screening of therapeutically useful reagents. A transgenic animal (e.g., a mouse or rat) is an animal having cells that contain a transgene. The transgene was introduced into the animal or an ancestor of the animal at a prenatal, e.g., an embryonic stage., A transgene is a piece of DNA which is integrated into the genome of a cell from which a transgenic animal develops. In one embodiment, cDNA encoding osteolevin can be used to clone genomic DNA encoding osteolevin protein (including the osteolevin allele containing the 3 base pair insertion at position 10668 defined by EMBL ACCESSION NO. AC003098) in accordance with established techniques. The genomic fragment can then be used to generate transgenic animals that contain cells which express DNA encoding osteolevin (for example a murine osteolevin protein having the 3 base pair insertion at position 10668 defined by EMBL ACCESSION NO. AC003098 seen in the human protein). Methods for generating transgenic animals, particularly animals such as mice or rats, have become conventional in the art and are described, for example, in U.S. Pat. Nos. 4,736,866 and 4,870,009. Typically, particular cells would be targeted for osteolevin transgene incorporation with tissue-specific enhancers. However, transgenic animals may also by generated in which the osteolevin transgene is expressed ubiquitously, for example under the control of the beta-actin promoter. Transgenic animals that include a copy of a transgene encoding various osteolevin sequences introduced into the germ line of the animal at an embryonic stage can be used to examine the effect of increased expression of DNA encoding osteolevin sequences. Such animals can be used as tester animals for reagents thought to confer protection from, for example, pathological conditions associated with its overexpression. In accordance with this facet of the invention, if transgenic animals treated with the reagent exhibit a reduced incidence of the pathological condition compared to untreated transgenic animals, then the reagent would be a candidate for potential therapeutic intervention for the pathological condition.

Alternatively, non-human homologues of osteolevin can be used to construct an osteolevin “knock out” animal. The knockout animal has a defective or altered gene in the osteolevin region as a result of homologous recombination between the endogenous gene encoding osteolevin region sequences and altered genomic DNA encoding osteolevin sequences introduced into an embryonic cell of the animal. For example, cDNA encoding osteolevin can be used to clone genomic DNA encoding osteolevin in accordance with established techniques. A portion of the genomic DNA encoding osteolevin can be deleted or replaced with another gene such as a gene encoding a selectable marker which can be used to monitor integration. Typically, several kilobases of unaltered flanking DNA (both at the 5′ and 3′ ends) are included in the vector (see e.g., Thomas and Capecchi, Cell, 51:503 (1987) for a description of homologous recombination vectors). The vector is introduced into an embryonic stem cell line (e.g., by electroporation) and cells in which the introduced DNA has homologously recombined with the endogenous DNA are selected (see e.g., Li et al., Cell 69:915 (1992)). The selected cells are then injected into a blastocyst of an animal (e.g., a mouse or rat) to form aggregation chimeras (see e.g., Bradley, in Teratocarcinomas and Embryonic Stem Cells.— A Practical Approach, E. J. Robertson, ed. (IRL, Oxford, 1987), pp. 113-152). A chimeric embryo can then be implanted into a suitable pseudopregnant female foster animal and the embryo brought to term to create a “knock-out” animal. Progeny harboring the homologously recombined DNA in their germ cells can be identified by standard techniques and used to breed animals in which all cells of the animal contain the homologously recombined DNA. Knockout animals can be characterized for instance for their development of pathological conditions due to absence of the osteolevin polypeptide.

The methods and osteolevin sequences disclosed herein also provide for a variety of drug screening assays using recombinant osteolevin sequences, typically osteolevin sequences having one or more polymorphisms that are associated with a pathological condition. Such proteins are particularly useful in the pharmacological characterization of novel modulators (i.e. inhibitors or activators) of the activity of proteins and protein complexes. Moreover, a variety of drug screening assays are known in the art, the methods of which are readily adapted for evaluating the osteolevin sequences disclosed herein (see e.g. Vinggaard et al., Toxicol. Appl. Pharmacol. 155(2): 150-160 (1999); Femandes et al., Curr. Opin. Chem. Biol., 2(5): 597-603 (1998); Gonzales et al., Curr. Opin. Biotechnol. 9(6): 624-31 (1998) and U.S. Pat. Nos. 5,877,007 and 5,780,258, the contents of which are incorporated herein by reference).

In an illustrative embodiment of such assays, recombinant polymorphic osteolevin protein can be introduced into the media of cells which are known to respond to wild type osteolevin protein. The response may be quantified for example, by measuring alterations in the pH of the media with Cytosensor microphysiometer system (manufactured by Molecular Dynamics), or by measuring changes in intracellular calcium concentrations, cyclic AMP or GMP levels, phosphorylation of proteins on a signaling pathway, or by a reporter gene assay. Such responsive changes in a particular activity or other cellular characteristic may be utilized in many useful ways, including the discovery, development or characterization of substances suitable for the treatment of diseases or other conditions in human beings or animals. Such osteolevin proteins and associated cell assays may also be useful for studying diseases or other biological processes, for determining the effects of various drugs alone or in combination, as well as for identifying or characterizing substances which may be useful in reducing or preventing the occurrence of a disease or other condition.

A specific embodiment of the invention provides methods and compositions for screening for agents which regulate osteolevin activity or osteolevin-induced activity. Such agents can find use in modulating a wide variety of physiological manifestations of osteolevin expression including the pathologies which are associated with osteolevin polymorphisms.

A cell-based assay to test the pharmacological activity of various agents can then be performed by exposing cells that respond to osteolevin to a candidate agent under conditions where the presence of the agent may cause a change in the response of the cell to osteolevin. In a closely related embodiment, a comparison between the responses of cells to (1) mutant and (2) wild type osteolevin can be characterized.

The cells to produce recombinant osteolevin for the assay described herein may be generated from individuals having polymorphisms in the osteoevin region or by a variety of protocols that are well known in the art including the transfection methods described above. Alternatively such cells can be generated by utilizing transgenic or genetic knock-out animals made by homologous recombination, e.g. recombination of a wild type osteolevin sequence with a transgene comprising a polymorphic or modified osteolevin sequence. Similarly, the cells for the assay itself may be cells that endogenously express the osteolevin receptor or may have been transfected with the receptor. In addition, a wide variety of reporter genes and assays that are known in the art can be adapted to the cell-based screening assays disclosed herein. For example, a reporter gene can encode an enzyme which produces colorimetric or fluorometric change in the host cell which is detectable by in situ analysis and which is a quantitative or semi-quantitative function of transcriptional activation. Exemplary enzymes include esterases, phosphatases, proteases (tissue plasminogen activator or urokinase) and other enzymes capable of being detected by activity which generates a chromophore or fluorophore as will be known to those skilled in the art. A preferred example is E.coli beta-galactosidase disclosed herein. This enzyme produces a color change upon cleavage of the indigogenic substrate indolyl-B-D-galactoside by cells bearing beta-galactosidase (see, e.g., Goring et al., Science, 235:456-458 (1987) and Price et al., Proc. Natl. Acad. Sci. U.S.A., 84:156-160 (1987)). This enzyme is preferred because the endogenous beta-galactosidase activity in mammalian cells ordinarily is quite low, the analytic screening system using 6-galactosidase is not hampered by host cell background.

Regulators of osteolevin activity or osteolevin-induced activity as identified in the method described herein before are also object of the present invention as are pharmaceutical compositions comprising such regulators and a pharmaceutically acceptable carrier.

Medicaments containing an osteolevin polypeptide are also an object of the present invention, as is a process for the manufacture of such medicaments, which process comprises bringing osteolevin polypeptide and, if desired, one or more other therapeutically valuable substances into a galenical administration form. The pharmaceutical compositions may be administered orally, for example in the form of tablets, coated tablets, dragées, hard or soft gelatine capsules, solutions, emulsions or suspensions. Administration can also be carried out rectally, for example using suppositories; locally or percutaneously, for example using ointments, creams, gels or solutions; or parenterally, for example using injectable solutions.

For the preparation of tablets, coated tablets, dragees or hard gelatine capsules the compounds of the present invention may be admixed with pharmaceutically inert, inorganic or organic excipients. Examples of suitable excipients for tablets, dragees or hard gelatine capsules include lactose, maize starch or derivatives thereof, talc or stearic acid or salts thereof. Suitable excipients for use with soft gelatine capsules include for example vegetable oils, waxes, fats, semi-solid or liquid polyols etc.; according to the nature of the active ingredients it may however be the case that no excipient is needed at all for soft gelatine capsules. For the preparation of solutions and syrups, excipients which may be used include for example water, polyols, saccharose, invert sugar and glucose. For injectable solutions, excipients which may be used include for example water, alcohols, polyols, glycerine, and vegetable oils. For suppositories, and local or percutaneous application, excipients which may be used include for example natural or hardened oils, waxes, fats and semi-solid or liquid polyols. The pharmaceutical compositions may also contain preserving agents, solubilising agents, stabilising agents, wetting agents, emulsifiers, sweeteners, colorants, odorants, salts for the variation of osmotic pressure, buffers, coating agents or antioxidants. They may also contain other therapeutically valuable agents.

Therefore, in accordance with the invention, osteolevin polypeptides may be used for the manufacture of a medicament for the treatment of diseases associated with abnormal bone formation, such as sclerosteosis, Van Buchem's disease, Paget disease and the like. The dosage can vary within wide limits and is, of course, fitted to the individual requirements in each particular case.

In a further embodiment of the invention, there are provided articles of manufacture and kits containing probes, oligonucleotides or antibodies which can be used, for instance, for the diagnostic applications described above. The article of manufacture comprises a container with a label. Suitable containers include, for example, bottles, vials, and test tubes. The containers may be formed from a variety of materials such as glass or plastic. The container holds a composition which includes an agent that is effective for diagnostic applications, such as described above. The label on the container indicates that the composition is used for a specific diagnostic application. The kit of the invention will typically comprise the container described above and one or more other containers comprising materials desirable from a commercial and user standpoint, including buffers, diluents, filters and package inserts with instructions for use.

The present invention is further detailed in the following Examples, which are offered by way of illustration and are not intended to limit the invention in any manner. Standard techniques well known in the art or the techniques specifically described below are utilized. All patent and literature references cited in the present specification are hereby incorporated by reference in their entirety.

### EXAMPLES

Commercially available reagents referred to in the examples were used according to manufacturer's instructions unless otherwise indicated.

### Example 1

Cloning and Sequencing of Osteolevin cDNA

A cDNA library derived from human kidney tissue (Clontech) was screened by hybridization. The probe used was obtained by PCR, using primers derived from the predicted 3′ UTR of the osteolevin gene. Positive dones were grown and plasmid DNA was isolated using high pure Plasmid Isolation kit (Roche). Sequencing on an automated sequencing apparatus (ABI377) using vector primers and internal primers for the gene revealed the presence of four clones. Sequencing according to standard procedures revealed the full-length cDNA clone shown in FIG. 1B.

### Example 2

Detection of Polymorphisms

a) To detect polymorphisms the osteolevin gene was PCR-amplified from 47 unrelated individuals of 5 different ethnic origins. Using fragment-specific primer pairs (length: 18-27 bp), 200-700 bp fragments were amplified e.g. a 588 PCR product was generated with the primer pair OSTVprom-F2 (FIG. 5) and OSTVprom-R2 (FIG. 5). Fragments were designed overlapping and covered the whole genomic region of osteolevin. After a column purification of the PCR products, the DNA was sequenced on an ABI capillary sequencer using ABI Dye terminator chemistry (fluorescence based sequencing). Polymorphisms in the DNA sequences were detected using Polyphred software (Nickerson, D. et al. 1997: NAR 25(14): 2745-2751), which operates on the basis of Phred, Phrap and Consed (programs all licensed from the University of Washington, USA). This program is able to automatically detect the presence of heterozygous single nucleotide substitutions by fluorescence-based sequencing. In the example above the following 5 polymorphisms were detected in the 588 bp fragment:

| | |
|---|---|
| C to T | 10877 |
| A to G | 10876 |
| T to C | 10817 |
| C to A | 10687 |
| TCC insertion between | 10668 and 10669 |

* defined by the position in EMBL ACCESSION NO. AC003098

b) The three disease-associated polymorphisms were detected by PCR-amplification of the appropriate genomic DNA-fragment from Van Buchem's and scierosteosis patients, followed by sequencing of the fragments and comparison to DNA of healthy individuals. To make high throughput screening possible a PCR-based assay was developed for two mutations. For the mutation in the Brazilian patients, a modified primer was designed creating a MboI restriction site in the mutated allele. After MboI restriction digest and electrophoresis the mutated allele can be differentiated from the normal. The mutated allele in the American patients contains a BstEII restriction site that is absent in the wild type allele making differentiation between mutated and normal allele possible.

### Example 3

Expression of Recombinant Osteolevin

Three types of recombinant osteolevin were produced. The first has a carboxy terminal tag, the second an amino terminal tag, and the third has no tag. The epitope tags were incorporated for purification of the protein. Constructs were made by standard methods (cite Maniatis or Protocols in Molec-ular Biology here). Primers were designed to the appropriate region of the osteolevin sequence with restriction sites at the 5′ end of each sequence as detailed below. Human osteolevin sequence was amplified by PCR, the product digested with appropriate enzymes, and ligated into a vector which had been similarly digested.

The construct for expression of the carboxy-terminal tagged protein was made in the pBlueBac4.5/V5-His vector (Invitrogen) using the BamHI and HindIII sites. The entire osteolevin coding sequence was amplified for insertion into the vector, including the endogenous putative secretion signal. The vector includes a V5 epitope. This permits the expression of the fusion protein to be monitored by Western analysis with an anti-V5 epitope antibody (Invitrogen). In addition the vector encodes a His6 tag which allows purification of the polypeptide on a nickel column (available from many sources).

The construct for expression of an amino-terminal tagged protein was made in the pAcSecG2T vector (Pharmingen) using the BamHI and EcoRI sites. The putative mature osteolevin coding sequence (i.e. not including the signal sequence) was amplified for insertion into the vector. The vector itself encodes a secretion signal taken from the gp67 protein for high levels of secretion. Additionally, the vector includes a glutathione S-transferase tag which allows for both monitoring of expression with anti-GST antibody (Pharmingen) and purification with glutathione agarose beads (Pharmingen). Finally, there is a thrombin cleavage site between the GST tag and osteolevin so that the protein can be separated from the tag following purification

The construct for expression of an untagged protein was made in the pBlueBac4.5 vector (Invitrogen) using the BamHI and EcoRI sites. The entire coding sequence for osteolevin, including the putative signal sequence, was amplified for insertion into the vector.

Each construct was completely sequenced to verify that no nucleotide changes had been inadvertently introduced during the amplification or cloning process.

Transformation of suitable host cells, growing of the transformed cells as well as specific expression of osteolevin have been performed following the manufacturer's instructions.

1. A method for screening for osteolevin polymorphisms in a subject, said method comprising determining the presence of a polymorphism in the osteolevin nucleic acid sequence obtained from the subject.

2. A method according to claim 1, wherein the polymorphisms are associated with abnormal bone formation.

3. A method for screening for the presence of a heritably linked form of abnormal bone formation in a subject, comprising determining the presence of a polymorphism associated with abnormal bone formation in an osteolevin nucleic acid sequence obtained from the subject.

4. The method according to any of claims 1 or 3, wherein the polymorphism is characterized as an insertion, deletion, substitution or a repetitive nucleotide sequence.

5. The method according to any of claims 1 to 4, wherein the polymorphism effects the expression or function of the osteolevin protein.

6. The method according to any of claims 1 or 3, wherein the presence of a polymorphism in the osteolevin nucleic acid sequence is determined by an assay comprising a differential nucleic acid analysis technique such as restriction fragment length polymorphism analysis, direct mass-analysis of PCR products using mass spectrometry, direct analysis of invasive cleavage products, direct sequence analysis, extension based techniques such as ARMS M (amplification refractory mutation system), ALEXM (amplification refractory mutation system linear extension) and COPS (competitive oligonucleotide priming system), OLA (oligonucleotide ligation assay), Invader Assay, DNA chip analysis or polymerase chain reaction analysis.

7. The method according to claim 4, wherein a PCR product is amplified with a forward primer selected from the group consisting of OSTV3UT-F, OSTVint-F1, OSTVint-F3, OSTVint-F4, OSTVint-F5, OSTVexl-F, OSTV5UT-F, OSTV3gen-F1, OSTV3gen-F2, OSTV5UT-F, OSTVprom-F1 and OSTVprom-F2, and a reverse primer selected from the group consisting of OSTV3UT-R, OSTVint-R1, OSTVint-R3, OSTVint-R4, OSTVint-R5, OSTVexl-R, OSTV5UT-R, OSTV3gen-R1, OSTV3gen-R2, OSTV5UT-R, OSTVprom-R1 and OSTVprom-R2.

8. A method of identifying a patient's susceptibility to a pathology associated with abnormal bone formation comprising the step of determining a genetic polymorphism pattern for a osteolevin nucleic acid sequence obtained from the patient and comparing the patient's genetic polymorphism pattern and the wild type osteolevin sequence, wherein differences between the patient's genetic polymorphism pattern and the wild type sequence indicate a susceptibility to the pathology.

9. A method of identifying a polymorphism associated with abnormal bone formation in one or more subjects, comprising comparing a osteolevin sequence isolated from the subjects to a known wild type osteolevin sequence and identifying a recurrent polymorphism in the osteolevin sequence isolated from the subjects being associated with abnormal bone formation.

10. A nucleic acid molecule encoding osteolevin, said nucleic acid molecule being selected from the group consisting of
a) the nucleic acid molecule or a complement of the nucleic acid molecule set forth in SEQ ID NO. 1.
b) a nucleic acid molecule or a complement of a nucleic acid molecule encoding a polypeptide set forth in SEQ ID NO. 2.
c) a nucleic acid molecule capable of hybridizing to a nucleic acid molecule of above a) or b).

11. A nucleic acid molecule encoding osteolevin region polymorphisms comprising the nucleic acid as defined by nucleotide position 4'000-11'000 in EMBL ACCESSION NO. AC003098, Release 62.0, or parts thereof, with one or more of the nucleotide exchanges or insertion selected from the group consisting of:

| | |
|---|---|
| G to A | at position 6136 |
| C to T | at postion 6140 |
| T to A | at postion 9047 |
| C to T | at position 10877 |
| A to G | at position 10876 |
| T to C | at position 10817 |
| C to A | at position 10687 |
| TCC between | position 10668 and 10669 |
| C to G | at position 10424 |
| C to G | at position 10342 |
| A to G | at position 10020 |
| T to C | at position 9783 |
| C to T | at position 9723 |
| C to T | at position 9646 |
| G to A | at position 9616 |
| C to T | at position 9242 |
| A to G | at position 8375 |
| C to T | at position 7894 |
| G to T | at position 7489 |
| T to G | at position 6358 |
| C to T | at position 5308 |
| G to A | at position 5004 |
| C to T | at position 4866 |
| G to C | at position 4475 |

and combinations thereof as well as their reverse complements.

12. A vector, comprising a nucleic acid molecule according to claim 10 or 11.

13. An expression vector, comprising a nudeic acid molecule according to claim 10 or 11 operatively linked to a transcriptional regulatory sequence.

14. A host cell transfected with a vector of any of claims 12 or 13.

15. A recombinant osteolevin polypeptide encoded by a nucleic acid molecule of claim 10 or 11.

16. A compound according to claim 15 for use in the treatment of diseases associated with abnormal bone formation, such as sderosteosis, Van Buchem's disease, Paget disease and the like.

17. The use of a compound according to claim 15 for the manufacture of a medicament for the treatment of diseases that are associated with abnormal bone formation, such as sclerosteosis, Van Buchem's disease, Paget disease and the like.

18. A pharmaceutical composition comprising a pharmaceutically acceptable carrier and a pharmaceutically effective amount of a polypeptide as defined in claim 15.

19. A method of preparing a recombinant osteolevin polypeptide of claim 15, comprising the steps of
a) culturing the cell of claim 14 in an appropriate culturing medium to produce an osteolevin polypeptide; and
b) isolating the osteolevin polypeptide.

20. A method for screening for regulators of osteolevin activity or osteolevin-induced activity, said method comprising the step of:
a) determining the expression level of a reporter gene or downstream signaling molecule such as intracellular calcium which is modulated by osteolevin in a particular cell type,
b) contacting the cell with a candidate agent,
c) determining the expression level of the reporter gene or downstream signaling molecule in response to the candidate agent; and
d) comparing the first expression level of the reporter or the downstream signaling molecule with the expression level observed in response to the candidate agent, wherein an alteration indicates that the candidate agent modulates osteolevin activity.

21. An antibody which binds specifically to a polypeptide according to claim 15.

22. The antibody of claim 21, which is a monoclonal antibody.

23. The antibody of any of claims 21 to 22, labelled with a detectable label.

24. A kit for identifying polymorphism in the osteolevin region, comprising at least one reagent for use in characterizing a nucleic acid sequence in the osteolevin region, instructions setting forth a procedure according to any of the methods of claims 1 to 9, and a container for contents of the kit.

25. A kit according to claim 24, wherein the reagent for use in characterizing a nucleic acid sequence in the osteolevin region comprises a polynucleotide capable of specifically hybridizing to the osteolevin region.

26. A regulator of osteolevin activity or osteolevin-induced activity as identified in the method of claim 20.

27. A pharmaceutical composition comprising a regulator of claim 26 and a pharmaceutically acceptable carrier.

28. The invention as described herein before.
